(19) United States
(12) Patent Application Publication
(10) Pub. No.: US 2026/0199644 A1
(43) Pub. Date: Jul. 16, 2026

(54) Applications for Smart Inflation Device

(71) Applicant: St. Jude Medical, Cardiology Division, Inc., St. Paul, MN (US)

(72) Inventor: Jay Reimer, Shoreview, MN (US)

(73) Assignee: St. Jude Medical, Cardiology Division, Inc., St. Paul, MN (US)

(21) Appl. No.: 19/428,145

(22) Filed: Dec. 20, 2025

Related U.S. Application Data
(60) Provisional application No. 63/744,187, filed on Jan. 11, 2025.

(51) Int. Cl. A61M 25/10; A61B 17/00; A61B 17/22; A61B 17/32; A61B 17/3207; A61B 90/00

(52) U.S. Cl. CPC A61M 25/104; A61B 17/22; A61B 17/320758; A61B 90/06; A61M 25/10181; A61M 25/10187; A61B 2017/00022; A61B 2017/00398; A61B 2017/00539; A61B 2017/00544; A61B 2017/00557; A61B 2017/00778; A61B 2017/22001; A61B 2017/22062; A61B 2017/22065; A61B 2017/320004; A61B 2090/064; A61M 2205/3327; A61M 2205/3331; A61M 2210/12

(57) ABSTRACT

A system for disrupting calcium within a blood vessel includes a handle, a balloon catheter extending from the handle, a balloon positioned at a distal end portion of the balloon catheter, and a fluid reservoir in fluid communication with a lumen, the lumen being in fluid communication with an interior volume of the balloon. The system also includes a motor operably coupled to the fluid reservoir, and a pressure sensor fluidically in contact with the fluid reservoir and configured to sense a pressure within the balloon. The motor is configured to alternately (i) push fluid from the fluid reservoir through the lumen toward the interior volume of the balloon and (ii) withdraw fluid from the interior volume of the balloon toward the fluid reservoir, so as to cycle between balloon inflation and deflation to repeatedly press the balloon against an interior surface of the blood vessel.

### CROSS-REFERENCE TO RELATED APPLICATIONS

This application claims priority to U.S. Provisional Patent Application No. 63/744,187, filed Jan. 11, 2025, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves include surgical heart valves, as well as collapsible and expandable heart valves intended for transcatheter aortic valve replacement or implantation (“TAVR” or “TAVI”) or transcatheter mitral valve replacement (“TMVR”). Surgical or mechanical heart valves may be sutured into a native annulus of a patient during an open-heart surgical procedure, for example. Collapsible and expandable heart valves may be delivered into a patient via a delivery apparatus such as a catheter to avoid a more invasive procedure such as full open-chest, open-heart surgery. As used herein, reference to a “collapsible and expandable” heart valve includes heart valves that are formed with a small cross-section that enables them to be delivered into a patient through a catheter in a minimally invasive procedure, and then expanded to an operable state once in place, as well as heart valves that, after construction, are first collapsed to a small cross-section for delivery into a patient and then expanded to an operable size once in place in the valve annulus.

Whereas known balloon-expandable prosthetic heart valve systems typically use a manually controlled indeflator or syringe-type device to inflate and deflate the balloon of the balloon catheter, it may be advantageous to use a motorized balloon inflation system with smart functionality. Such balloon inflation systems, while useful for balloon-expandable TAVR procedure, could have dual use (or alternative use) in other procedures as well. The present disclosure addresses problems and limitations associated with the related art.

### SUMMARY OF THE DISCLOSURE

According to one aspect of the disclosure, a system for disrupting calcium within a blood vessel (e.g. a vein, an artery, or any other vessel that carries blood) includes a handle, a balloon catheter extending from the handle, a balloon positioned at a distal end portion of the balloon catheter, and a fluid reservoir in fluid communication with a lumen, the lumen being in fluid communication with an interior volume of the balloon. The system also includes a motor operably coupled to the fluid reservoir, and a pressure sensor fluidically in contact with the fluid reservoir and configured to sense a pressure within the balloon. The motor is configured to alternately (i) push fluid from the fluid reservoir through the lumen toward the interior volume of the balloon and (ii) withdraw fluid from the interior volume of the balloon toward the fluid reservoir, so as to cycle between balloon inflation and deflation to repeatedly press the balloon against an interior surface of the blood vessel. The pressure sensor may be positioned interior to the balloon. During operation of the motor to cycle between balloon inflation and deflation, a processor operably coupled to the pressure sensor may be configured to monitor pressure data received from the pressure sensor to determine a maximum cycle pressure within the balloon during each successive cycle of balloon inflation and deflation. During operation of the motor to cycle between balloon inflation and deflation, the processor may be configured to compare each determined maximum cycle pressure to a threshold pressure value and to indicate that successful calcium disruption has occurred when one of the determined maximum cycle pressures is smaller than the threshold pressure value. The threshold pressure value may be set to a fraction of a maximum value of all of the determined maximum cycle pressures. The threshold pressure value may be set to a fraction of the determined maximum cycle pressure within the balloon during a first one of the cycles of balloon inflation and deflation.

According to another aspect of the disclosure, a method of disrupting calcium within a blood vessel (e.g. a vein, an artery, or any other vessel that carries blood) includes inserting a balloon catheter into a patient's vasculature, the balloon catheter being connected to a handle, a balloon being positioned at a distal end portion of the balloon catheter, a fluid reservoir being fluidically coupled to an interior volume of the balloon via a lumen. The balloon catheter may be advanced to a treatment site within the blood vessel while the balloon is in a deflated condition. While the balloon catheter is at the treatment site, a motor of a fluid delivery system may be actuated to alternately (i) push fluid from the fluid reservoir through the lumen toward the interior volume of the balloon to inflate the balloon into contact with an interior surface of the blood vessel and (ii) withdraw fluid from the interior volume of the balloon toward the fluid reservoir to deflate the balloon, so as to cycle between balloon inflation and deflation to repeatedly press the balloon against the interior surface of the blood vessel to disrupt the calcium at the treatment site. A pressure sensor may be fluidically in contact with the fluid reservoir, and the method may include monitoring pressure within the balloon over time during the cycling between balloon inflation and deflation. A maximum cycle pressure within the balloon may be determined during each successive cycle of balloon inflation and deflation. Each determined maximum cycle pressure within the balloon may be compared to a threshold pressure value. Comparing each determined maximum cycle pressure within the balloon to the threshold pressure value may be performed by visually comparing a plot of the monitored pressure within the balloon over time, provided on a display, to the threshold pressure value. Comparing each determined maximum cycle pressure within the balloon to the threshold pressure value may be performed by a processor operably coupled to the fluid delivery system. Cycling between balloon inflation and deflation may be performed at least until the determined maximum cycle pressure within the balloon is lower than the threshold pressure value. The threshold pressure value may be set to a fraction of a maximum value of all of the determined maximum cycle pressures. The threshold pressure value may be set to a fraction of the determined maximum cycle pressure within the balloon during a first one of the cycles of balloon inflation and deflation.

According to a further aspect of the disclosure, a system for disrupting calcium within a blood vessel (e.g. a vein, an artery, or any other vessel that carries blood) includes a handle, a catheter extending from the handle, an abrasive element positioned at a distal end portion of the catheter, a drive shaft coupled to the abrasive element so that rotation of the drive shaft causes rotation of the abrasive element, a rotational drive mechanism coupled to the drive shaft so that rotation of the rotational drive mechanism causes rotation of the drive shaft, and a fluid reservoir in fluid communication with a lumen, the lumen being in fluid communication with the rotational drive mechanism so that advancing fluid from the fluid reservoir through the lumen causes rotation of the rotational drive mechanism. The system also includes a motor operably coupled to the fluid reservoir, and a pressure sensor fluidically in contact with the fluid reservoir and configured to sense a pressure within the fluid reservoir. The motor is configured to push fluid from the fluid reservoir through the lumen to the rotational drive mechanism to rotate the abrasive element. The pressure sensor may be positioned interior to the fluid reservoir. During operation of the motor to rotate the abrasive element, a processor operably coupled to the pressure sensor may be configured to monitor pressure data received from the pressure sensor.

According to a further aspect of the disclosure, a method of disrupting calcium and/or plaque within a blood vessel (e.g. a vein, an artery, or any other vessel that carries blood) includes inserting a catheter into a patient's vasculature, the catheter being connected to a handle, an abrasive element being positioned at a distal end portion of the catheter, a drive shaft coupled to the abrasive element, a rotational drive mechanism coupled to the drive shaft, and a fluid reservoir being fluidically coupled to the rotational drive mechanism via a lumen. The catheter may be advanced to a treatment site within the blood vessel. While the catheter is at the treatment site, a motor of a fluid delivery system is actuated to push fluid from the fluid reservoir through the lumen to the rotational drive mechanism to rotate (i) the rotational drive mechanism, (ii) the drive shaft, and (iii) the abrasive element to disrupt the calcium and/or plaque. During rotation of the abrasive element, a pressure sensor is fluidically in contact with the fluid reservoir, the method including monitoring pressure within the fluid reservoir based on pressure data received from the pressure sensor. The pressure sensor may be positioned interior to the fluid reservoir.

### BRIEF DESCRIPTION OF THE DRAWINGS

FIG. 1 is a perspective view of an example of a prosthetic heart valve.

FIG. 2 is a front view of an example of a section of the frame of the prosthetic heart valve of FIG. 1, as if cut longitudinally and laid flat on a table.

FIG. 3 is a front view of an example of a prosthetic leaflet of the prosthetic heart valve of FIG. 1, as if laid flat on a table.

FIG. 4 is a top view of the prosthetic heart valve of FIG. 1 mounted on an example of a portion of a delivery system.

FIG. 5 is an enlarged view of the example of the handle of the delivery system shown in FIG. 4.

FIG. 6 is an enlarged view of an example of the distal end of the delivery system shown in FIG. 4.

FIG. 7 is a top view of an example of a balloon catheter when the balloon is inflated.

FIG. 8 is a top view of an example of an inflation system for use with a delivery system similar to that shown in FIG. 4.

FIG. 9 is a side view of the example of the inflation system of FIG. 8.

FIG. 10 is a perspective view of an example of a connection between the inflation system of FIGS. 8-9 and the handle of the delivery system of FIG. 4.

FIG. 11 is a flowchart showing exemplary steps in a procedure to implant the prosthetic heart valve of FIG. 1 into a patient using the delivery system of FIG. 4.

FIG. 12A is a perspective view of an example of a bottom enclosure of a smart balloon inflation system.

FIG. 12B is a perspective view of an example of a motor or engine housing of a smart balloon inflation system.

FIG. 12C is a front perspective view of an example of a top enclosure of a smart balloon inflation system.

FIG. 12D is a rear perspective view of the top enclosure of FIG. 12C.

FIG. 12E is a perspective view of an example of a syringe of a smart balloon inflation system.

FIGS. 12F-12I are various views of an alternate version of the enclosure(s) of FIGS. 12A, 12C, and 12D.

FIG. 13A is a perspective view of the bottom enclosure of FIG. 12A and the motor or engine housing of FIG. 12B in an assembled condition.

FIG. 13B is an isolated rear perspective view of the top enclosure of FIGS. 12C-12D assembled to the assembly of FIG. 13A.

FIG. 14 is a top view of a smart balloon inflation system formed by assembling the components of FIGS. 12A-12E.

FIG. 15A is a perspective view of an example of a syringe.

FIG. 15B is a first cutaway view of the syringe of FIG. 15A.

FIG. 15C is a second cutaway view of the syringe of FIG. 15B.

FIG. 16A is a schematic view of an example of a balloon inflation system interacting with other components useful in a prosthetic heart valve implantation procedure.

FIG. 16B is an example of a balloon compliance curve illustrating pressure versus volume of the balloon of FIG. 16A.

FIG. 16C is an exemplary display screen during a planning step of a prosthetic heart valve implantation.

FIG. 16D is an exemplary display screen during a mid-procedural step of a prosthetic heart valve implantation.

FIG. 16E is an exemplary display screen showing a speed profile selection option as a part of a prosthetic heart valve implantation.

FIG. 16F is a side view of an example of a prosthetic heart valve delivery system handle with a feature for staged inflation speeds.

FIG. 16G is a side view of another example of a prosthetic heart valve delivery system handle with an alternative feature for staged inflation speeds.

FIGS. 17A-17C are exemplary display screens showing a post dilatation procedure.

FIG. 18 is a schematic view of an example of a de-airing setup using the delivery system of FIG. 4.

FIGS. 19A-H are schematic illustrations of steps in an exemplary calcium disruption procedure using a smart balloon inflation system.

FIG. 19I illustrates a graph of pressure versus time, showing an exemplary pressure waveform generated from the cycle of inflation and deflation of FIGS. 19A-19H.

FIG. 20A is a perspective view of a rotational atherectomy device.

FIG. 20B is a highly schematic view of the rotational atherectomy device of FIG. 20A coupled to a smart inflation system.

FIG. 21 is a block diagram that illustrates an example of a computer system upon which an example may be implemented.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing valves disclosed herein. However, it should be noted that the use of the valve is not limited to the intended position and orientation but may be deployed in any type of lumen or passageway. For example, although prosthetic heart valves are described herein as prosthetic aortic valves, those same or similar structures and features can be employed in other heart valves, such as the pulmonary valve, the mitral valve, or the tricuspid valve. Further, the term “proximal,” when used in connection with a delivery device or system, refers to a position relatively close to the user of that device or system when it is being used as intended, while the term “distal” refers to a position relatively far from the user of the device. In other words, the leading end of a delivery device or system is positioned distal to the trailing end of the delivery device or system, when the delivery device is being used as intended. As used herein, the terms “substantially,” “generally,” “approximately,” and “about” are intended to mean that slight deviations from absolute are included within the scope of the term so modified. As used herein, the prosthetic heart valves may assume an “expanded state” and a “collapsed state,” which refer to the relative radial size of the stent.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (known as paravalvular or “PV” leakage).

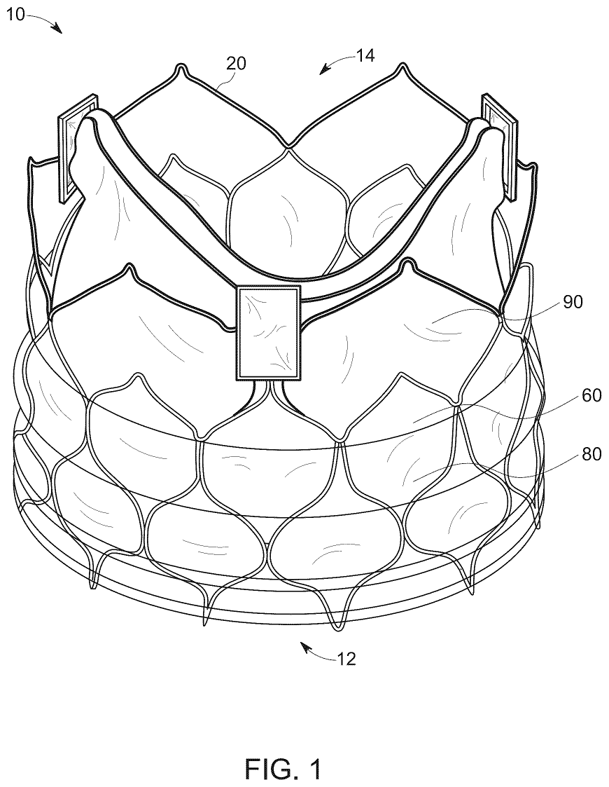

FIG. 1 is a perspective view of one example of a prosthetic heart valve 10. Prosthetic heart valve 10 may be a balloon-expandable prosthetic aortic valve, although in other examples it may be a self-expandable or mechanically-expandable prosthetic heart valve, intended for replacing a native aortic valve or another native heart valve. Prosthetic heart valve 10 is shown in an expanded condition in FIG. 1. Prosthetic heart valve 10 may extend between an inflow end 12 and an outflow end 14. Prosthetic heart valve 10 may include a collapsible and expandable frame 20, an inner cuff or skirt 60, an outer cuff or skirt 80, and a plurality of prosthetic leaflets 90. As should be clear below, prosthetic heart valve 10 is merely one example of a prosthetic heart valve, and other examples of prosthetic heart valves may be suitable for use with the concepts described below.

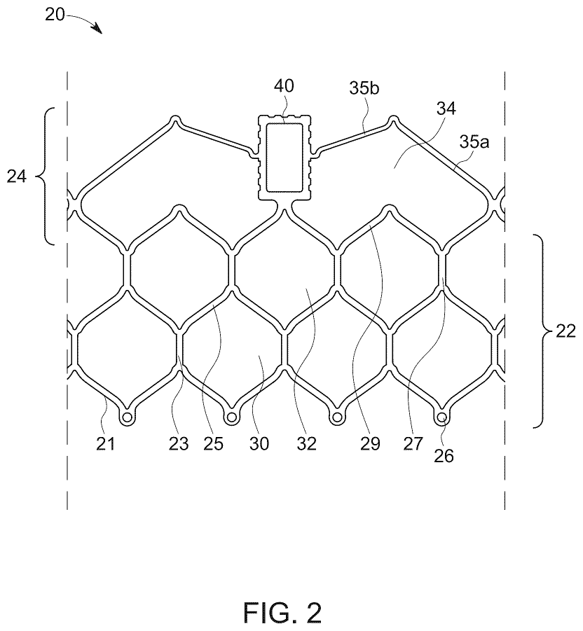

Now referring in addition to FIG. 2., FIG. 2 is a front view of an example of a section of the frame 20 of prosthetic heart valve 10, as if cut longitudinally and laid flat on a table. The section of frame 20 in FIG. 2 may represent approximately one-third of a complete frame, particularly if frame 20 is used in conjunction with a three-leaflet prosthetic heart valve. In the illustrated example, frame 20 is a balloon-expandable stent and may be formed of stainless steel or cobalt-chromium, and which may include additional materials such as nickel and/or molybdenum. However, in some embodiments the stent may be formed of a shape memory material such as nitinol or the like. The frame 20, when provided as a balloon-expandable frame, is configured to collapse upon being crimped to a smaller diameter and/or expand upon being forced open, for example via a balloon within the frame expanding, and the frame will substantially maintain the shape to which it is modified when at rest.

Frame 20 may include an inflow section 22 and an outflow section 24. The inflow section 22 may also be referred to as the annulus section. In one example, the inflow section 22 includes a plurality of rows of generally hexagon-shaped cells. For example, the inflow section 22 may include an inflow-most row of hexagon-shaped cells 30 and an outflow-most row of hexagon-shaped cells 32. The inflow-most row of hexagonal cells 30 may be formed of a first circumferential row of angled or zig-zag struts 21, a second circumferential row of angled or zig-zag struts 25, and a plurality of axial struts 23 that connect the two rows. In other words, each inflow-most hexagonal cell 30 may be formed by two angled struts 21 that form an apex pointing in the inflow direction, two angled struts 25 that form an apex pointing in the outflow direction, and two axial struts that connect the two angled struts 21 to two corresponding angled struts 25. The outflow-most row of hexagonal cells 32 may be formed of the second circumferential row of angled or zig-zag struts 25, a third circumferential row of angled or zig-zag struts 29, and a plurality of axial struts 27 that connect the two rows. In other words, each outflow-most hexagonal cell 32 may be formed by two angled struts 25 that form an apex pointing in the inflow direction, two angled struts 29 that form an apex pointing in the outflow direction, and two axial struts that connect the two angled struts 27 to two corresponding angled struts 29. It should be understood that although the term “outflow-most” is used in connection with hexagonal cells 32, additional frame structure, described in more detail below, is still provided in the outflow direction relative to the outflow-most row of hexagonal cells 32.

In the illustrated embodiment, assuming that frame 20 is for use with a three-leaflet valve and thus the section shown in FIG. 2 represents about one-third of the frame 20, each row of cells 30, 32 includes twelve individual cells. However, it should be understood that more or fewer than twelve cells may be provided per row of cells. Further, the inflow or annulus section 22 may include more or fewer than two rows of cells. Still further, although cells 30, 32 are shown as being hexagonal, the some or all of the cells of the inflow section 22 may have other shapes, such as diamond-shaped, chevron-shaped, or other suitable shapes. In the illustrated embodiment, every cell 30 in the first row is structurally similar or identical to every other cell 30 in the first row, every cell 32 in the second row is structurally similar or identical to every other cell 32 in the second row, and every cell 30 in the first row is structurally similar or identical (excluding the aperture 26) to every cell 32 in the second row. However, in other examples, the cells in each row are not identical to every other cell in the same row or in other rows.

An inflow apex of each hexagonal cell 30 may include an aperture 26 formed therein, which may accept sutures or similar features which may help couple other elements, such as an inner cuff 60, outer cuff 80, and/or prosthetic leaflets 90, to the frame 20. However, in some examples, one or more or all of the apertures 26 may be omitted.

Still referring to FIG. 2, the outflow section 24 of the frame 20 may include larger cells 34 that have generally asymmetric shapes. For example, the lower or inflow part of the larger cells 34 may be defined by the two upper struts 29 of a cell 32, and one upper strut 29 of each of the two adjacent cells 32. In other words, the lower end of each larger cell 34 may be formed by a group of four consecutive upper struts 29 of three circumferentially adjacent cells 32. The tops of the larger cells 34 may each be defined by two linking struts 35a, 35b. The first linking strut 35a may couple to a top or outflow apex of a cell 32 and extend upwards at an angle toward a commissure attachment feature (“CAF”) 40. The second linking strut 35b may extend from an end of the first linking strut 35a back downwardly at an angle and connect directly to the CAF 40. To the extent that the larger cells 34 include sides, a first side is defined by a portion of the CAF 40, and a second side is defined by the connection between first linking strut 35a and the corresponding upper strut 29 of the cell 32 attached to the first linking strut 35a.

The CAF 40 may generally serve as an attachment site for leaflet commissures (e.g. where two prosthetic leaflets 90 join each other) to be coupled to the frame 20. In the illustrated example, the CAF 40 is generally rectangular and has a longer axial length than circumferential width. The CAF 40 may define an interior open rectangular space. The struts that form CAF 40 may be generally smooth on the surface defining the open rectangular space, but some or all of the struts may have one or more suture notches on the opposite surfaces. For example, in the illustrated example, CAF 40 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets 90 to the frame 20, and/or may directly couple an intermediate sheet of material (e.g. fabric or tissue) to the CAF 40, with the prosthetic leaflets 90 being directly coupled to that intermediate sheet of material. In some embodiments, tabs or ends of the prosthetic leaflets 90 may be pulled through the opening of the CAF 40, but in other embodiments the prosthetic leaflets 90 may remain mostly or entirely within the inner diameter of the frame 20. It should be understood that balloon-expandable frames are typically formed of metal or metal alloys that are very stiff, particularly in comparison to self-expanding frames. At least in part because of this stiffness, although the prosthetic leaflets 90 may be sutured or otherwise directly coupled to the frame at the CAFs 40, it may be preferable that most or all of the remaining portions of the prosthetic leaflets 90 are not attached directly to the frame 20, but are rather attached directly to an inner skirt 60, which in turn is directly connected to the frame 20. Further, it should be understood that other shapes and configurations of CAFs 40 may be appropriate. For example, various other suitable configurations of frames and CAFs are described in greater detail in U.S. patent application Ser. No. 18/810,994, filed Aug. 21, 2024 and titled “TAVI Deployment Accuracy-Stent Frame Improvements,” the disclosure of which is hereby incorporated by reference herein.

With the example described above, frame 20 includes two rows of hexagon-shaped cells 30, 32, and a single row of larger cells 34. In a three-leaflet embodiment of a prosthetic heart valve that incorporates frame 20, each row of hexagon-shaped cells 30, 32 includes twelve cells, while the row of larger cells includes six larger cells 34. As should be understood, the area defined by each individual cell 30, 32 is significantly smaller than the area defined by each larger cell 34 when the frame 20 is expanded. There is also significantly more structure (e.g. struts) that create each row of individual cells 30, 32 than structure that creates the row of larger cells 34.

One consequence of the above-described configuration is that the inflow section 22 has a higher cell density than the outflow section 24. In other words, the total numbers of cells, as well as the number of cells per row of cells, is greater in the inflow section 22 compared to the outflow section 24. The configuration of frame 20 described above may also result in the inflow section 22 being generally stiffer than the outflow section 24 and/or more radial force being required to expand the inflow section 22 compared to the outflow section 24, despite the fact that the frame 20 may be formed of the same metal or metal alloy throughout. This increased rigidity or stiffness of the inflow section 22 may assist with anchoring the frame 20, for example after balloon expansion, into the native heart valve annulus. The larger cells 34 in the outflow section 24 may assist in providing clearance to the coronary arteries after implantation of the prosthetic heart valve 10. For example, after implantation, one or more coronary ostia may be positioned above the frame 20, for example above the valley where two adjacent larger cells 34 meet (about halfway between a pair of circumferentially adjacent CAFs 40). Otherwise, one or more coronary ostia may be positioned in alignment with part of the large interior area of a larger cell 34 after implantation. Either way, blood flow to the coronary arteries is not obstructed, and a further procedure that utilizes the coronary arteries (e.g. coronary artery stenting) will not be obstructed by material of the frame 20. Still further, the lower rigidity of the frame 20 in the outflow section 24 may cause the outflow section 24 to preferentially foreshorten during expansion, with the inflow section 22 undergoing a relatively smaller amount of axial foreshortening. This may be desirable because, as the prosthetic heart valve 10 expands, the position of the inflow end of the frame 20 may remain substantially constant relative to the native valve annulus, which may make the deployment of the prosthetic heart valve 10 more precise. This may be, for example, because the inflow end of the frame 20 is typically used to gauge proper alignment with the native valve annulus prior to deployment, so axial movement of the inflow end of the frame 20 relative to the native valve annulus during deployment may make precise placement more difficult.

Referring back to FIG. 1, the prosthetic heart valve 10 may include an inner skirt 60 mounted to the interior surface of frame 20. The inner skirt 60 may be formed of tissue, such as pericardium, although other types of tissue may be suitable. In the illustrated example, the inner skirt 60 is formed of a woven synthetic fabric, such as polyethylene terephthalate (“PET”) or polytetrafluoroethylene (“PTFE”), although other fabrics may be suitable, including fabrics other than woven fabrics. In some examples, the inner skirt 60 has straight or zig-zag shaped inflow and outflow ends that generally follow the contours of the cells 30, 32 of the inflow section 22 of frame 20. Preferably, inner skirt 60 is sutured to the frame 20 along the struts that form cells 30, 32. If apertures 26 are included, inner skirt 60 may also be coupled to frame 20 via sutures passing through apertures 26. Preferably, the inner skirt 60 does not cover (or does not cover significant portions of) the larger cells 34. The inner skirt 60 may be coupled to the frame 20 via mechanisms other than sutures, including for example ultrasonic welding or adhesives. Further, the inner skirt 60 may have shapes other than that shown, and need not have a zig-zag inflow or outflow end, and need not cover every cell in the inflow section 22. In fact, in some examples, the inner skirt 60 may be omitted entirely, with the outer skirt 80 (described in greater detail below) being the only skirt used with prosthetic heart valve 10. If the inner skirt 60 is provided, it may assist with sealing the prosthetic heart valve 10 within the heart, as well as serving as a mounting structure for the prosthetic leaflets 90 (described in greater detail below) within the frame 20.

Still referring to FIG. 1, the prosthetic heart valve 10 may include an outer skirt 60 mounted to the exterior surface of frame 20. The outer skirt 80 may be formed of tissue, such as pericardium, although other types of tissue may be suitable. In the illustrated example, the outer skirt 80 is formed of a woven synthetic fabric, such as PET or PTFE, although other fabrics may be suitable, including fabrics other than woven fabrics. In some examples, the outer skirt 80 has straight or zig-zag inflow end. Preferably, outer skirt 80 is sutured to the frame 20 and/or inner skirt 60 along the inflow edge of the outer skirt 80. If apertures 26 are included, outer skirt 80 may also be coupled to frame 20 via sutures passing through apertures 26. The outer skirt 80 may include a plurality of folds or pleats, such a circumferentially extending folds or pleats. The folds or pleats may be formed in the outer skirt 80 via heat setting, for example by placing the outer skirt 80 within a mold that forces the outer skirt 80 to form folds of pleats, and the outer skirt 80 may be treated with heat so that the outer skirt 80 tends to maintain folds or pleats in the absence of applied forces. The outflow edge of outer skirt 80 may be coupled to the frame 20 at selected, spaced apart locations around the circumference of the frame 20. In some embodiments, the outflow edge of outer skirt 80 may be connected to the inner skirt 60 along a substantially continuous suture line. Some or all of the outer skirt 80 between its inflow and outflow edges may remain not directly couples to the frame 20 or inner skirt 60. Preferably, the outer skirt 80 does not cover (or does not cover significant portions of) the larger cells 34. In use, the outer skirt 80 may directly contact the interior surface of the native heart valve annulus to assist with sealing, including sealing against PV leak. If folds or pleats are included with the outer skirt 80, the additional material of the folds or pleats may help further mitigate PV leak. However, it should be understood that the folds or pleats may be omitted from outer skirt 80, and the outer skirt 80 may have shapes other than that shown. In fact, in some examples, the outer skirt 80 may be omitted entirely, with the inner skirt 60 being the only skirt used with prosthetic heart valve 10. If the inner skirt 60 is omitted, the prosthetic leaflets 90 may be attached directly to the frame 20 and/or directly to the outer skirt 80.

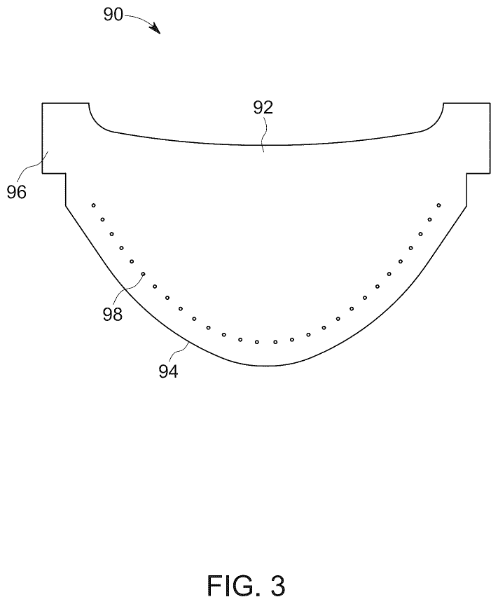

Now referring in addition to FIG. 3., FIG. 3 is a front view of an example of a prosthetic leaflet 90, as if laid flat on a table. In the illustrated example of prosthetic heart valve 10, a total of three prosthetic leaflets 90 are provided, although it should be understood that more or fewer than three prosthetic leaflets may be provided in other example of prosthetic heart valves. The prosthetic leaflet 90 may be formed of a synthetic material, such a polymer sheet or woven fabric, or a biological material, such a bovine or porcine pericardial tissue. However, other materials may be suitable. In on example, the prosthetic leaflet 90 is formed to have a concave free edge 92 configured to coapt with the free edges of the other leaflets to help provide the one-way valve functionality. The prosthetic leaflet 90 may include an attached edge 94 which is attached (e.g. via suturing) to other structures of the prosthetic heart valve 10. For example, the attached edge 94 may be coupled directly to the inner skirt 60, directly to the frame 20, and/or directly to the outer skirt 80. It may be preferable that the attached edge 94 is coupled directly only to the inner skirt 60, which may help reduce stresses on the prosthetic leaflet 90 compared to if the attached edge 94 were coupled directly to the frame 20. In some embodiments, a plurality of holes 98 may be formed along the attached edge 94 (or a spaced distance therefrom), for example via lasers. If included, the holes 98 may be used to receive sutures therethrough, which may make it easier to couple the prosthetic leaflet 90 to the inner skirt 60 during manufacturing. For example, the holes 98 may serve as guides if suturing is performed manually, and if the positions of the holes 98 are controlled via the use of layers, the holes 98 may be consistently placed among different prosthetic leaflets 90 to reduce variability between different prosthetic leaflets 90. Laflet tabs 96 may be provided at the junctions between the free edge 92 and the attached edge 94. Each leaflet tab 96 may be joined to a leaflet tab of an adjacent prosthetic leaflet to form prosthetic leaflet commissures, which may be coupled to the frame 20 via CAFs 40.

The prosthetic heart valve 10 may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing or supporting the valve is inserted through the femoral artery and advanced against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

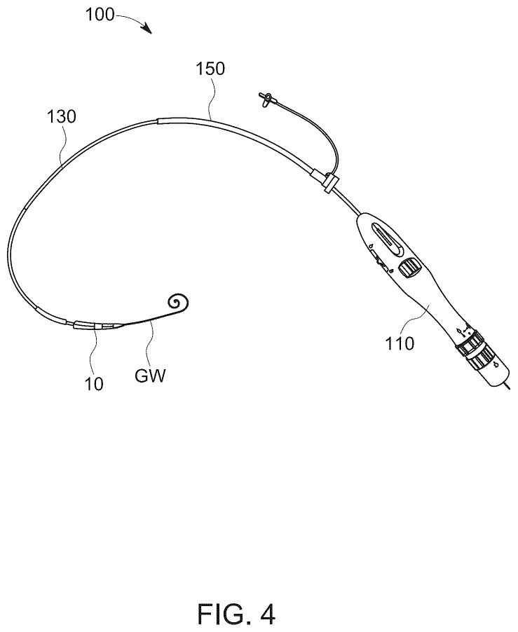

Now referring in addition to FIG. 4, FIG. 4 illustrates one example of a delivery system 100, with the prosthetic heart valve 10 crimped over a balloon on a distal end of the delivery system 100. Although delivery system 100 and various components thereof are described below, it should be understood that delivery system 100 is merely one example of a balloon catheter that may be appropriate for use in delivering and deploying prosthetic heart valve 10.

In some examples, delivery system 100 includes a handle 110 and a delivery catheter 130 extending distally from the handle 110. An introductory of 150 may be provided with the delivery system 100. Introducer 150 may be an integrated or captive introducer, although in other embodiments introducer 150 may be a non-integrated or non-captive introducer. In some examples, the introducer 150 may be an expandable introducer, including for example an introducer that expands locally as a large diameter components passes through the introducer, with the introducer returning to a smaller diameter once the large diameter components passes through the introducer. In other examples, the introducer 150 is a non-expandable introducer.

A guidewire GW may be provided that extends through the interior of all components of the delivery system 100, from the proximal end of the handle 110 through the atraumatic distal tip 138 of the delivery catheter 130. The guidewire GW may be introduced into the patient to the desired location, and the delivery system 100 may be introduced over the guidewire GW to help guide the delivery catheter 130 through the patient's vasculature over the guidewire GW.

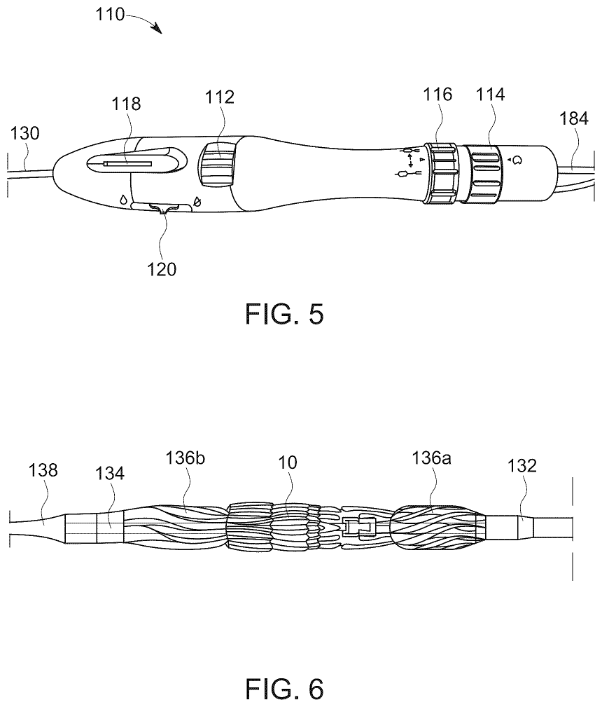

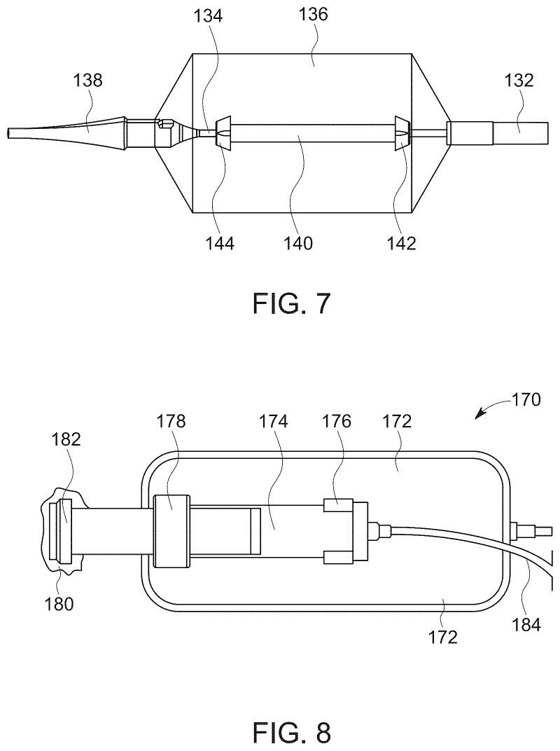

In some examples, the delivery catheter 130 is steerable. For example, one or more steering wires may extend through a wall of the delivery catheter 130, with one end of the steering wire coupled to a steering ring coupled to the delivery catheter 130, and another end of the steering wire operable coupled to a steering actuator on the handle 110. In such examples, as the steering actuator is actuated, the steering wire is tensioned or relaxed to cause deflection or straightening of the delivery catheter 130 to assist with steering the delivery catheter 130 to the desired position within the patient. For example, FIG. 5 is an enlarged view of the handle 110. Handle 110 may include a steering knob 112 that, upon rotation, tensions or relaxes the steering wires to deflect the distal end of the delivery catheter 130. Handle 110 may include a slot 118 with an indicator extending therethrough, the indicator moving along the slot 118 as the delivery catheter 130 deflects (e.g. the indicator moves proximally as deflection increases). If included, the indicator and slot 118 may provide the user an easy reference of how much the delivery catheter 130 is deflected at any given point. However, it should be understood that the steering functionality may be omitted in some examples, and in other examples steering actuators other than knobs may be utilized. Further, in some examples, including those shown in FIGS. 6-7, the delivery catheter 130 includes an outer catheter 132, and an inner catheter 134. The inner catheter 134 may also be referred to as a guidewire catheter. The steering functionality may be provided in either the outer catheter 132, or the inner catheter 134, or in both catheters. However, in some examples, a separate steering catheter 135 may be provided. For example, as shown in FIG. 4, the steering catheter 135 may be positioned outside of the outer catheter 132 and may terminate just proximal to the balloon 136. With this configuration, deflection of the steering catheter 135 will also cause deflection of the outer catheter 132 and the inner catheter 134 which are both nested within the steering catheter 135.

Still referring to FIGS. 4-5, the delivery system 100 may include additional functionality to assist with positioning the prosthetic heart valve 10. For example, in the illustrated example, handle 110 includes a commissure alignment actuator 114, which may be positioned near a proximal end of the handle or at any other desired location. In the illustrated example, the commissure alignment actuator 114 is in the form of a rotatable knob, although other forms may be suitable. The commissure alignment knob 114 may be rotationally coupled to a portion of the delivery catheter 130 supporting the prosthetic heart valve 10. For example, the commissure alignment actuator 114 may be rotationally coupled to an inner catheter 134 which supports the prosthetic heart valve 10 in the crimped condition. With this configuration, rotating the commissure alignment knob 114 may cause the inner catheter 134 to rotate about its longitudinal axis, and thus cause the prosthetic heart valve 10 to rotate about its longitudinal axis. If a commissure alignment actuator 114 is included, it may be used to help ensure that, upon deployment of the prosthetic heart valve 10 into the native valve annulus, the commissures of the prosthetic heart valve are in rotational alignment with respective ones of the native valve commissures (e.g. within +/−2.5 degrees of rotational alignment, within +/−5 degrees of rotational alignment, within +/−10 degrees of rotational alignment, within +/−15 degrees of rotational alignment, etc.). Although commissure alignment actuator 114 is shown in this example as a knob positioned at or near a proximal end of the handle 110, it should be understood that the actuator 114 may take forms other than a knob, may be positioned at other suitable locations, and may be omitted entirely if desired.

Still referring to FIGS. 4-5, the delivery system 100 may include even further functionality to assist with positioning the prosthetic heart valve 10. For example, in the illustrated example, handle 110 includes an axial alignment actuator 116, which may be positioned near a proximal end of the handle, including distal to the commissure alignment actuator 114, or at any other desired location. In the illustrated example, the axial alignment actuator 116 is in the form of a rotatable knob, although other forms may be suitable. The axial alignment knob 116 may be operably coupled to a portion of the delivery catheter 130 supporting the prosthetic heart valve 10. For example, the axial alignment actuator 116 may include internal threads that engage external threads of a carriage that is coupled to an inner catheter 134 which supports the prosthetic heart valve 10 in the crimped condition. In such an example, the carriage may be rotatably fixed to the handle 110. With this configuration, rotating the axial alignment knob 116 may cause the carriage to advance distally or retract proximally as the inner threads of the axial alignment knob 116 mesh with the external threads of the carriage, but the carriage is prevented from rotating. As the carriage advances distally or retracts proximally, the inner catheter 134 may correspondingly advance distally or retract proximally, and thus cause the prosthetic heart valve 10 to advanced distally or retract proximally. It should be understood that, if axial alignment actuator 116 is included, it have a small total range of motion. In other words, the rough or coarse axial alignment between the prosthetic heart valve 10 and native valve annulus may be achieved by physically advancing the entire delivery catheter 130 by pushing it through the vasculature while holding the handle 110. However, for fine and more controlled adjustment of the axial position of the prosthetic heart valve 10 relative to the native valve annulus, which may be performed just prior to or during deployment of the prosthetic heart valve 10, the axial alignment knob 116 may be used. If an axial alignment actuator 116 is included, it may be used to help ensure that, upon deployment of the prosthetic heart valve 10 into the native valve annulus, the inflow end of the of the prosthetic heart valve is in axial alignment with the inflow aspect of the native valve annulus (e.g. within +/−0.5 mm of axial alignment, within +/−1.0 mm of axial alignment, within +/−1.5 mm of axial alignment, within +/−2.0 mm of axial alignment, etc.). Although axial alignment actuator 116 is shown in this example as a knob positioned at or near a proximal end of the handle 110, it should be understood that the actuator 116 may take forms other than a knob, may be positioned at other suitable locations, and may be omitted entirely if desired.

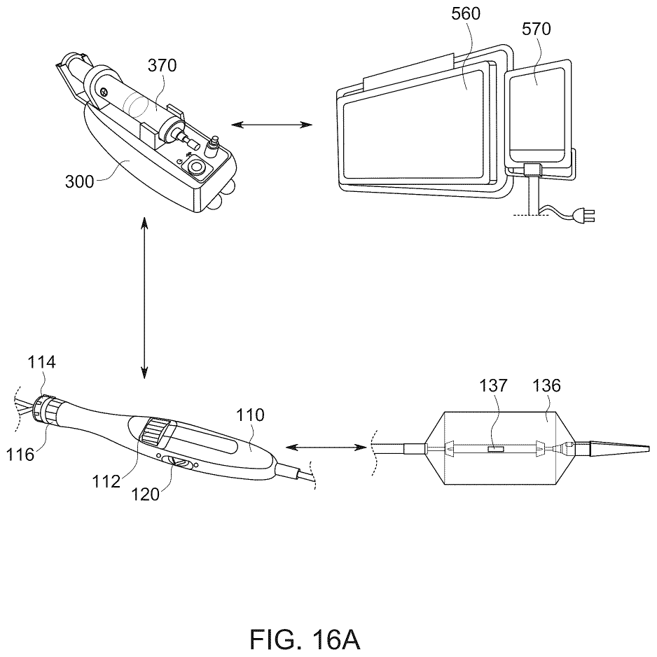

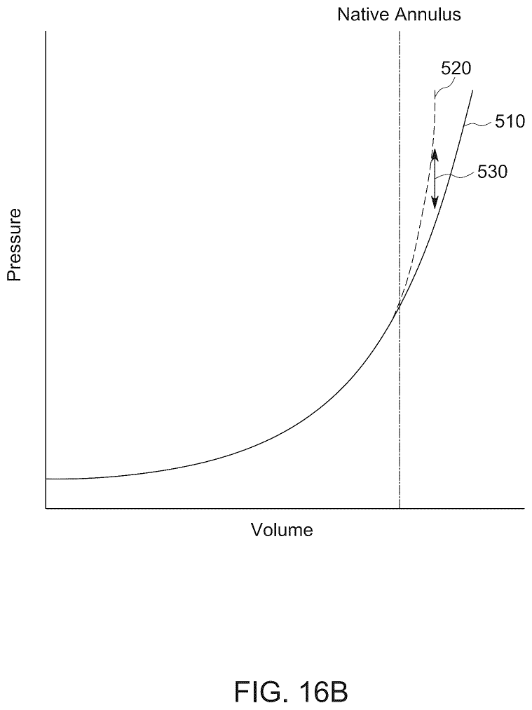

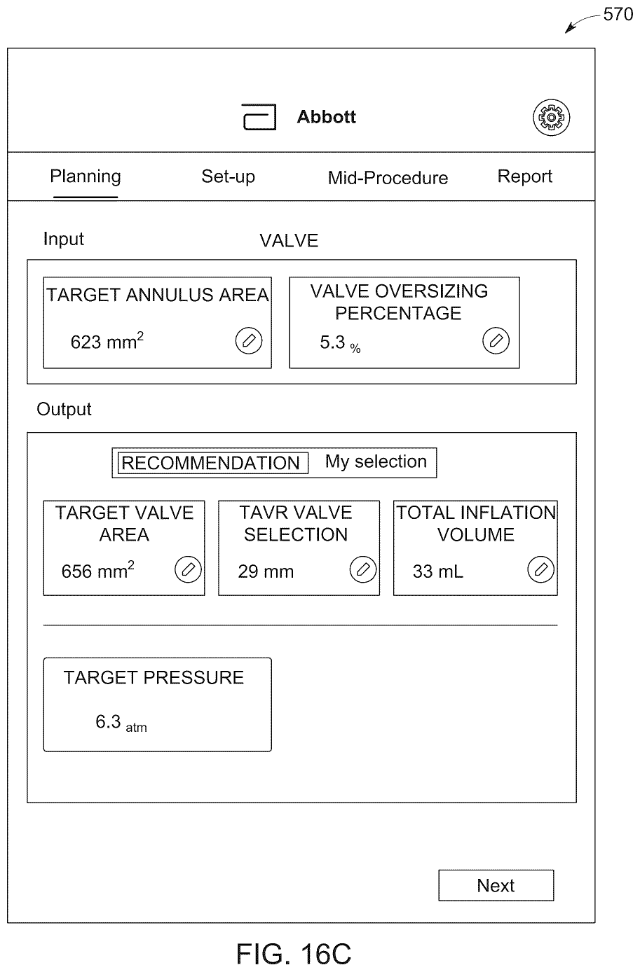

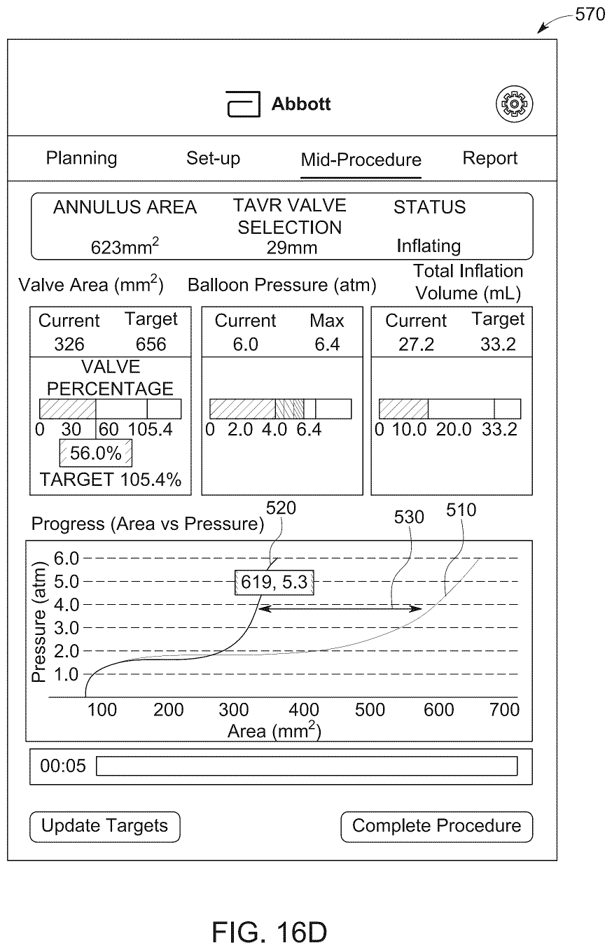

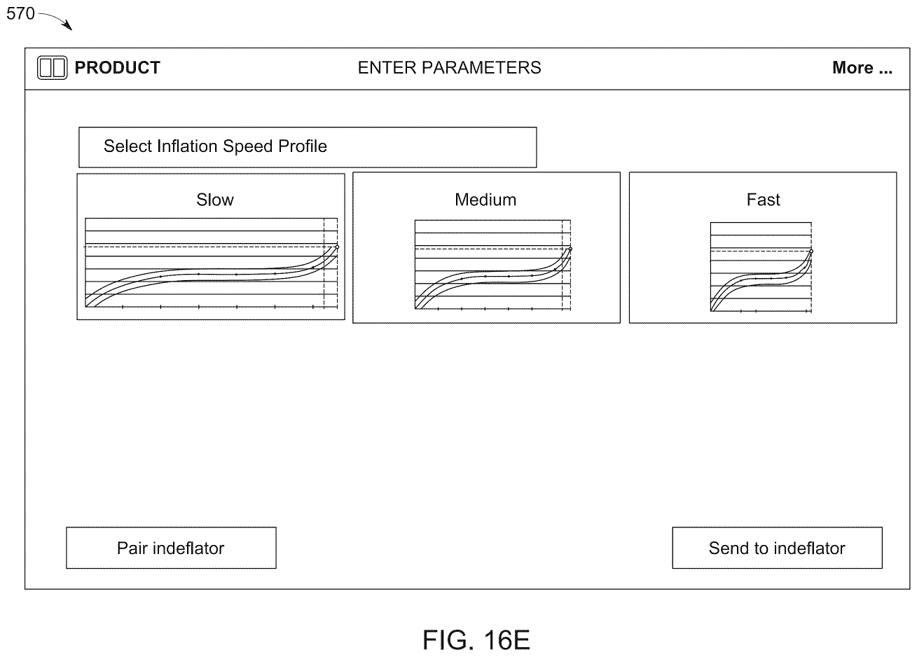

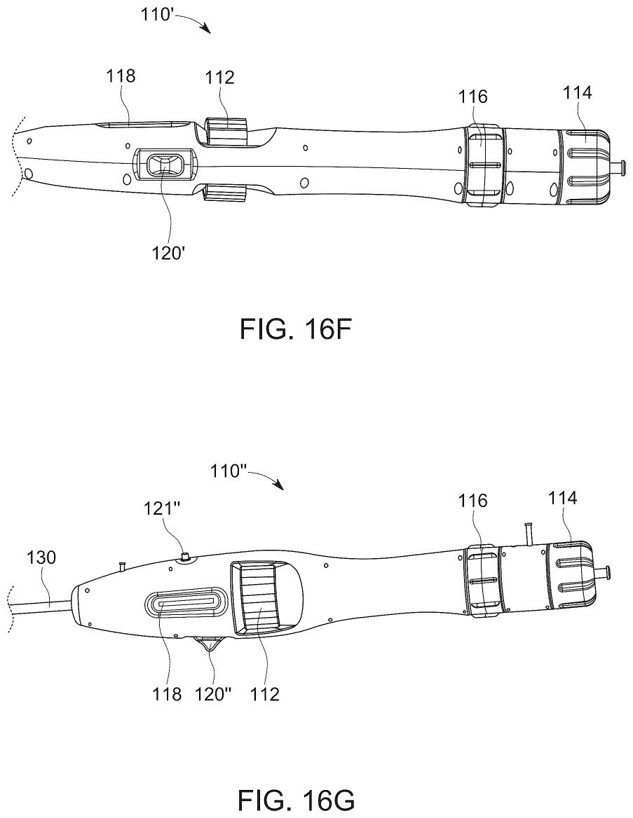

In addition to steering and positioning actuators, delivery system 100 may include a balloon actuator 120. Balloon actuator 120 may be an input device, for example similar to those described in connection with FIG. 16, that sends instructions to inflation system 170 or another device configured to control inflation. In the illustrated example, balloon actuator 120 is positioned on the handle 110 near a distal end thereof, and is provided in the form of a switch. Balloon actuator 120 may be actuated to cause inflation or deflation of a balloon 136 that is part of the delivery system 100. For example, referring briefly to FIGS. 6-7, the delivery system 100 may include a balloon 136 that overlies a distal end of inner catheter 134 and which receives the prosthetic heart valve 10 in a crimped condition thereon. In the example illustrated in FIG. 6, the balloon 136 includes a proximal pillowed portion 136a, a distal pillowed portion 136b, and a central portion over which the prosthetic heart valve 10 is crimped. The proximal pillow 136a and the distal pillow 136b may form shoulders on each side of the prosthetic heart valve 10, which may help ensure the prosthetic heart valve 10 does not move axially relative to the balloon 136 and/or inner catheter 134 during delivery. The shoulder formed by the distal pillow 136 may also help protect the inflow edge of the prosthetic heart valve 10 from contact with the anatomy during delivery. For example, during a transfemoral delivery, as the distal end of the delivery catheter 130 traverse the sharp bends of the aortic arch (or during initial introduction into the patient), there is a relatively high likelihood the inflow end of the prosthetic heart valve 10 (which is the leading edge during transfemoral delivery) will contact a vessel wall (or a components of an introduction system) causing dislodgment of the prosthetic heart valve 10 relative to the balloon 136. The distal pillow 136 may tend to have an equal or larger outer diameter than the inflow end of the prosthetic heart valve 10 (when the prosthetic heart valve 10 is crimped and the balloon 136 is deflated), which may help ensure the inflow edge of the prosthetic heart valve 10 does not inadvertently contact another structure during delivery. In some examples, the pillowed portions 136a, 136b may be formed via heat setting. Additional related features for use in similar balloon catheter delivery systems are described in greater detail in U.S. Patent Application Publication No. 2024/0148501, the disclosure of which is hereby incorporated by reference herein.

In order to deploy the prosthetic heart valve 10, the balloon 136 is inflated, for example by actuating the balloon actuator 120 to force fluid (such as saline, although other fluids, including liquids or gases, could be used) into the balloon 136 to cause it to expand, causing the prosthetic heart valve 10 to expand in the process. For example, the balloon actuator 120 may be pressed forward or distally to cause fluid to travel through an inflation lumen within delivery catheter 130 to inflate the balloon 136. FIG. 7 illustrates an example of the balloon 136 after being inflated, with the prosthetic heart valve 10 omitted from the figure for clarity. In the illustrated example, the balloon 136 may be formed to have a distal end that is fixed to a portion of an atraumatic distal tip 138. The distal tip 138 may be tapered to help the delivery catheter 130 move through the patient's vasculature more smoothly. A proximal end of the balloon 136 may be fixed to a distal end of outer catheter 132. The inflation lumen may be the space between the outer catheter 132 and the inner catheter 134, or in other embodiments may be provided in a wall of the inner catheter 134, or in any other location that fluidly connects the interior of the balloon 136 to a fluid source outside of the patient that is operable coupled to the delivery system 100.

Now referring in addition to FIG. 7, in some examples, a mounting shaft 140 may be provided on the inner catheter 134. A proximal stop 142 and/or a distal stop 144 may be provided, for example at opposite ends of the mounting shaft 140. If the mounting shaft 140 is included, it may provide a location on which the prosthetic heart valve 10 may be crimped. If the proximal stop 142 and/or distal stop 144 is provided, they may provide physical barriers to the prosthetic heart valve 10 moving axially relative to the balloon 136. In one example, the proximal stop 142 may taper from a larger distal diameter to a smaller proximal diameter, and the distal stop may taper from a larger proximal diameter to a smaller distal diameter. The spacing between the proximal stop 142 and the distal stop 144, if both are included, may be slightly larger than the length of the prosthetic heart valve 10 when it is crimped over mounting shaft 140. However, it should be understood that one or both of the stops 142, 144 may be omitted, and the mounting shaft 140 may also be omitted. If the mounting shaft 140 is included, it is preferably axially and rotationally fixed to the inner catheter 134 so that movement of the inner catheter 134 causes corresponding movement of the mounting member 140, and thus the prosthetic heart valve 10 when mounted thereon.

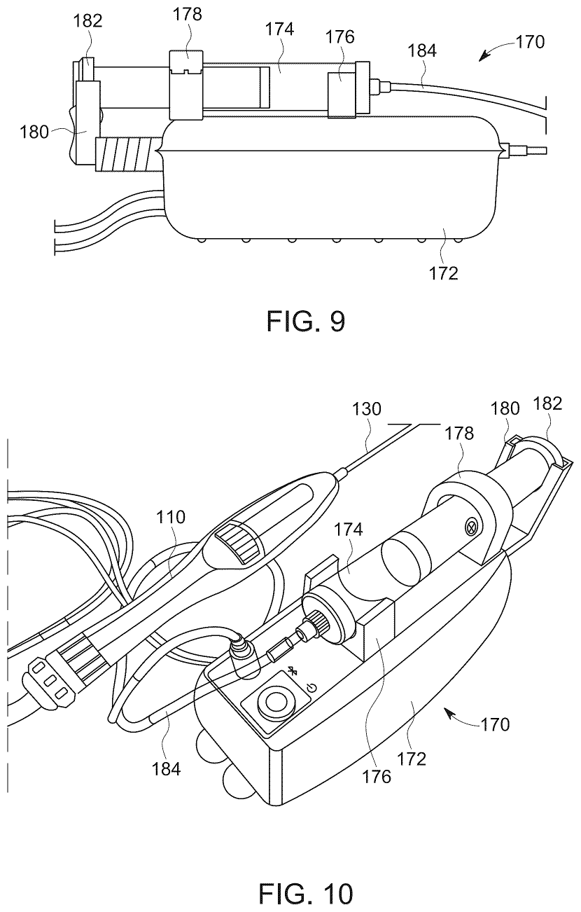

Before describing the use of balloon actuator 120 in more detail, it should be understood that in some embodiments, the balloon actuator 120 may be omitted and instead a manual device, such as a manual syringe, may be provided along with delivery system 100 in order to manually push fluid into balloon 136 during deployment of the prosthetic heart valve 10. However, in the illustrated example of delivery system 100, the balloon actuator 120 provides for a motorized and/or automated (or semi-automated) balloon inflation functionality. For example, FIG. 8 and FIG. 9 illustrate an example of a balloon inflation system 170. Balloon inflation system 170 may include a housing 172 that houses one or more components, which may include a motor, one or more batteries, electronics for control and/or communication with other components, etc. In some embodiments, the inflation system 170 forms a computer system, or components thereof, such as shown and described in connection with FIG. 16. Housing 172 may include one or more fixed cradles to receive a syringe 174. In the illustrated embodiment, a distal cradle 176 is provide with an open “C”- or “U”-shaped configuration so that the distal end of the syringe 174 may be snapped into or out of the distal cradle 176. A proximal cradle 178 may also be provided, which may have a “C”- or “U”-shaped bottom portion hingedly connected to a “C”- or “U”-shaped top portion. This configuration may allow for the proximal end of the outer body of the syringe 174 to be snapped into the bottom portion of proximal cradle 178, and the top portion of proximal cradle 178 may be closed and connected to the bottom portion to fully circumscribe the outer body of the syringe 174 to lock the syringe 174 to the housing 172. It should be understood that more or fewer cradles, of similar or different designs, may be included with housing 172 to help secure the syringe 174 to the housing 172 in any suitable fashion.

The balloon inflation system 170 may include a moving member 180. In the illustrated embodiment, moving member 180 includes a “C”- or “U”-shaped cradle to receive a plunger handle 182 of the syringe 174 therein, the cradle being attached to a carriage that extends at least partially into the housing 172. The carriage of the moving member 180 may be generally cylindrical, and may include internal threading that mates with external threading of a screw mechanism (not shown) within the housing 172 that is operably coupled to a motor. In some embodiments, the carriage may have the general shape of a “U”-beam with the flat face oriented toward the top. The moving member 180 may be rotationally fixed to the housing 172 via any desirable mechanism, so that upon rotation of the screw mechanism by the motor, the moving member 180 advances farther into the housing 172, or retracts farther away from the housing 172, depending on the direction of rotation of the screw mechanism. While the plunger handle 182 is coupled to the moving member 180, advancement of the moving member 180 forces fluid from the syringe 174 toward the balloon 136, while retraction of the moving member 180 withdraws fluid from the balloon 136 toward the syringe 174. It should be understood that the motor, or other driving mechanism, may be located in or outside the housing 172, and any other suitable mechanism may be used to operably couple the motor or other driving mechanism to the moving member 180 to allow for axial driving of the plunger handle 182.

As shown in the examples of each of FIG. 8, FIG. 9, and FIG. 10, the distal end of syringe 174 may be coupled to tubing 184 that is in fluid communication with an inflation lumen of delivery catheter 130 that leads to the balloon 136 at or near the distal end of the delivery system 100. Tubing 184 may allow for the passage of the fluid (e.g., saline) from the syringe 174 toward the balloon 136, or for withdrawal of fluid from the balloon 136 toward the syringe 174, for example based on whether the balloon actuator 120 is pressed forward or backward.

Although not separately numbered in FIG. 8, FIG. 9, and FIG. 10, the housing 172 may include one or more cables extending from the housing, for example to allow for transmission of power (e.g. from AC mains or another component with which the cable is coupled) and/or transmission of data, information, control commands, etc. For example, one cable may couple the housing 172 to handle 110 so that controls on the handle 110 (e.g. balloon actuator 120) may be used to activate the balloon inflation system 170 in the desired fashion. Another cable may couple to a computer display or similar device to provide information regarding the inflation of the balloon 136. However, it should be understood that any transmission of data or information may be provided wirelessly instead of via a wired connection, for example via a Bluetooth or other suitable connection. Additional and related features of balloon inflation system 170, related systems, and the uses thereof are described in U.S. Patent Application Publication No. 2023/0372097, the disclosure of which is hereby incorporated by reference herein.

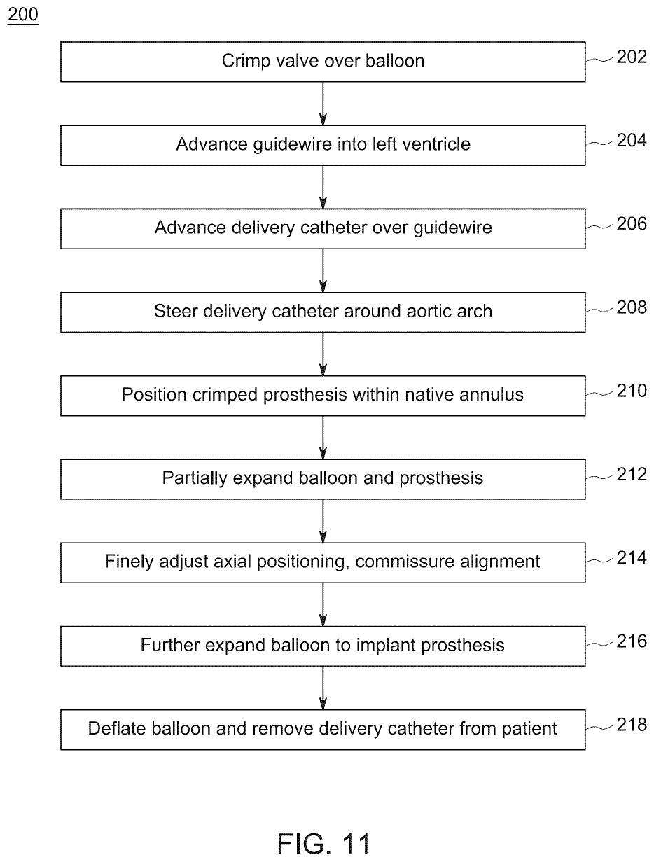

Now referring in addition to FIG. 11, FIG. 11 is a flowchart showing exemplary steps in an implantation procedure 200 to implant the prosthetic heart valve 10 of FIG. 1 into a patient using the delivery system 100 of FIG. 4. However, it should be understood that not all of the steps shown in connection with implantation procedure 200 need to be performed, and various steps not explicitly shown and described in connection with procedure 200 may be performed as part of the implantation procedure. At the beginning of the procedure 200 in step 202, the prosthetic heart valve 10 may be collapsed over or crimped onto balloon 136, with the balloon 136 being mostly or entirely deflated after the crimping procedure. It should be understood that crimping step 202 may be performed at any time prior to the procedure, including at the beginning of the procedure, or at an earlier stage before the delivery system 100 is provided to the end user. In other words, the crimping step 202 may be performed during a manufacturing stage of the delivery system 100 and/or prosthetic heart valve 10. During an early stage of the implantation procedure 200, a guidewire GW may be advanced into the patient in step 204, for example via the femoral artery, around the aortic arch, through the native aortic valve, and into the left ventricle. The guidewire GW may be used as a rail for other devices that need to access this pathway. For example, in step 206, the atraumatic distal tip 138 may be advanced over the proximal end of the guidewire GW, and the delivery catheter 130 may be advanced over guidewire GW toward the native aortic valve. During this initial advancement of the delivery catheter 130 into the patient, the introducer 150 (if included) may be positioned distally, for example so that it covers the prosthetic heart valve 10 or so that it is positioned just proximal to the prosthetic heart valve 10. Advancement of the delivery catheter 130 and introducer 150 may continue until a proximal hub of the introducer is in contact with the patient's skin (or in contact with another device that enters the patient's femoral artery. At this point, the introducer 150 may stop moving axially relative to the patient, with the delivery catheter 130 continuing to advance relative to the introducer 150. If steering capability is provided, the delivery catheter 130 may be steered or deflected at any point to assist with achieving the desired pathway of the delivery catheter 130. As on example, in step 208, the steering knob 112 may be actuated to deflect the distal end of the delivery catheter 130 as it traverses the sharp bends of the aortic arch. Advancement of the delivery catheter 130 may continue in step 210 until the prosthetic heart valve 10, while still crimped or collapsed, is positioned within the native aortic valve annulus. With the desired position achieved, the balloon 136 may be partially inflated, for example by pressing balloon actuator 120 forward, to partially expand the prosthetic heart valve 10 in step 212. In some examples, it is desirable to expand the prosthetic heart valve 10 only partially in step 212, because the position of the prosthetic heart valve 10 (including rotational and/or axial positioning) relative to the native aortic valve annulus may shift during this partial expansion. After the partial expansion of step 212, the user may examine the positioning of the prosthetic heart valve 10 relative to the native aortic valve annulus. If desired, in step 214, the axial positioning of the partially-expanded prosthetic heart valve 10 relative to the native aortic valve annulus may be finely adjusted (e.g. by actuating axial alignment actuator 116) and/or the rotational orientation of the prosthetic heart valve 10 relative to the native aortic valve may be finely adjust (e.g. by actuating commissure alignment actuator 114). When the desired axial alignment is achieve and the desired rotational alignment (e.g. rotational alignment between the prosthetic commissure and the native commissures) is achieved, the balloon 136 may be fully expanded in step 216 to fully expand the prosthetic heart valve 10 and to anchor the prosthetic heart valve 10 in the native aortic valve annulus in the desired position and orientation. After deployment is complete, the balloon 136 may be deflated in step 218, for example by pressing balloon actuator 120 backward, and the delivery catheter 130 and guidewire GW may be removed from the patient to complete the procedure. It should be understood that the nine steps shown in FIG. 11 as part of procedure 200 are merely exemplary of a single example of an implantation procedure, and steps shown may be omitted, steps not shown may be included, and steps may be provided in any order deemed appropriate by the physician and/or medical personnel.

Although various components of a prosthetic heart valve 10 and delivery system 100 are described above, it should be understood that these components are merely intended to provide better context to the systems, features, and/or methods described below. Thus, various components of the systems described above may be modified or omitted as appropriate without affecting the systems, features, and/or methods described below. For example, prosthetic heart valves other than the specific configuration shown and described in connection with FIGS. 1-3 may be used with delivery systems other than the specific configuration shown and described in connection with FIGS. 4-10 as part of an implantation procedure that uses steps other than the specific configuration shown and described in connection with FIG. 11, without affecting the inventive systems, features, and/or methods described below.

Reiterating certain points that have been described above, typical balloon-expandable heart valves are deployed into the native valve annulus by manually pushing fluid (e.g., saline) from a syringe to inflate a balloon and to expand the prosthetic heart valve into the native annulus. The reliance on fully manual balloon inflation may not be optimal, and it may be desirable to have partial or complete automation of the balloon inflation process, for example to provide more consistent and predictable results of the balloon expansion. For example, the predictability of how a balloon expandable prosthetic heart valve expands can vary greatly depending on how quickly the user inflates the balloon. Such systems may also be able to assist in providing data that can be used during the procedure, and which may also be collected among many procedures to learn information relating to important parameters of the balloon inflation that may not be otherwise easily determined from the typical manual process. This information may be gathered and used to refine the partial or fully automated balloon expansion process for future procedures. It would also be desirable for balloon inflation systems (or accessory components thereof) to be able to reliably de-air the balloon catheter system prior to use, preferably with an objective mechanism (e.g., other than only by eyesight) by which to confirm that no more than an acceptable amount of air remains in the catheter prior to delivery. In fact, a single smart inflation system may be capable of providing enhanced user experience throughout multiple phases of a procedure, including during preparation (e.g., de-airing), deployment of the valve, and post-dilatation after valve deployment (if desired). Still further, it may be useful to allow for such a smart inflation system to have features that assist with manual operation, if such manual operation becomes desirable or necessary (e.g., as a result of a power or other system failure). Some features of a smart inflation system are described in greater detail in U.S. Patent Application Publication No. 2023/0372097 (“the '097 Publication”), the disclosure of which his hereby incorporated by reference herein.

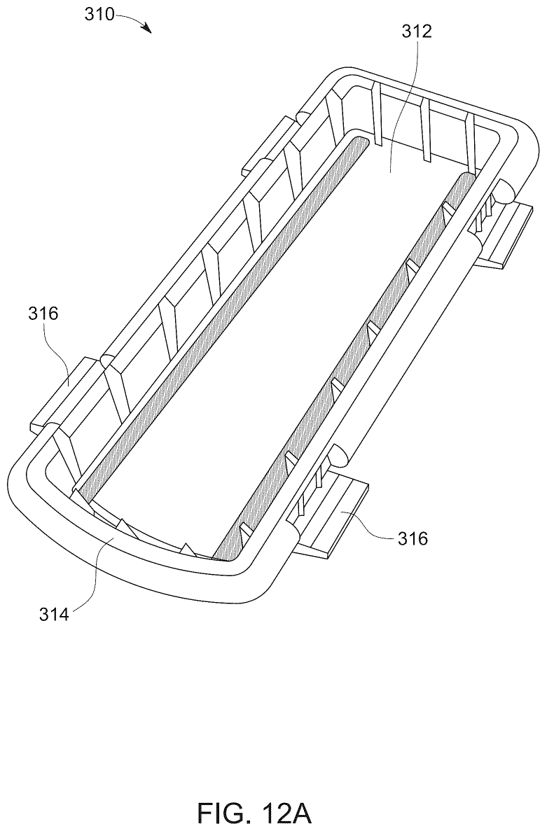

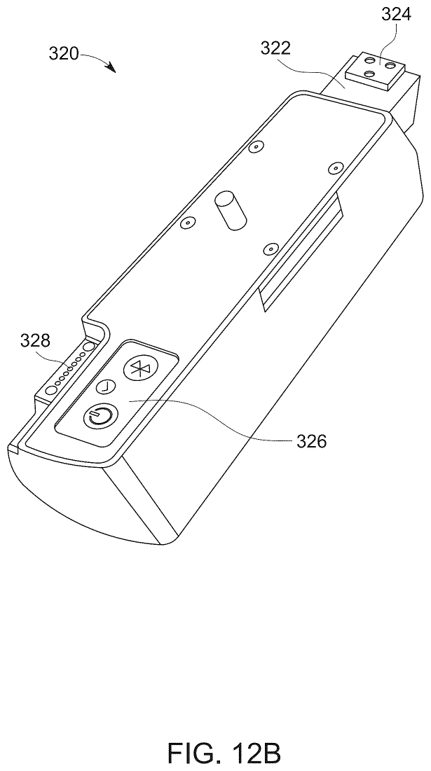

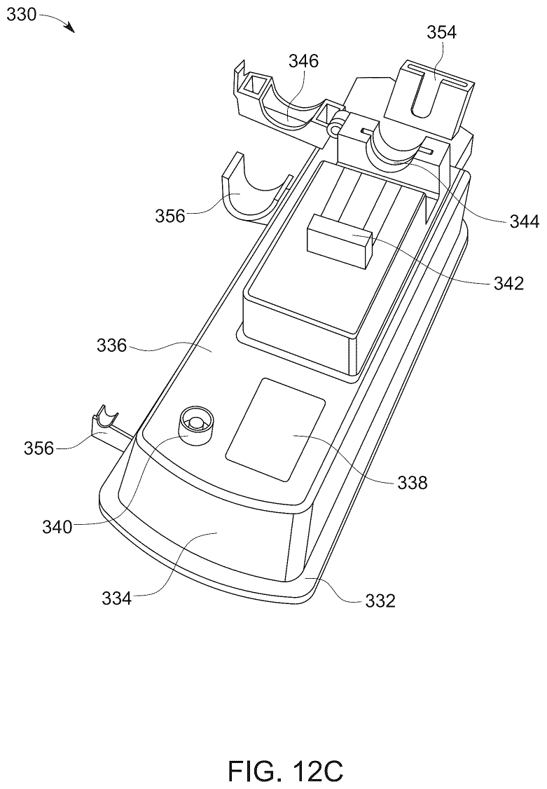

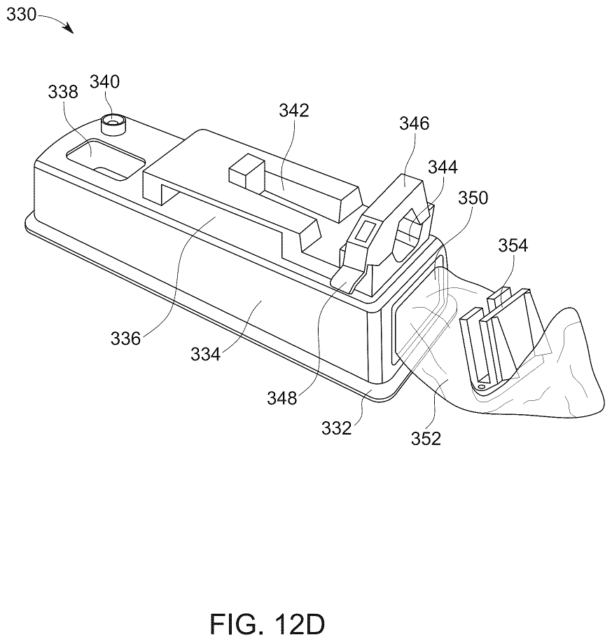

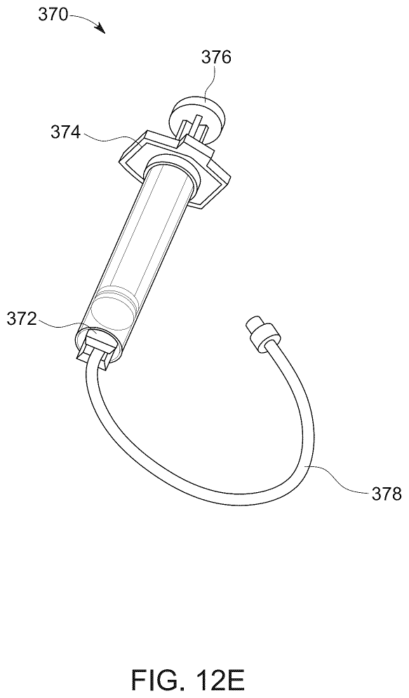

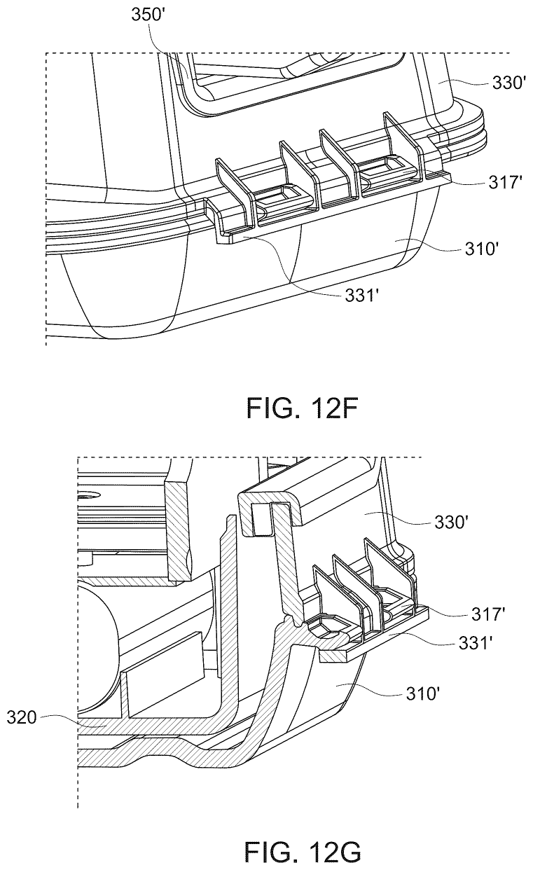

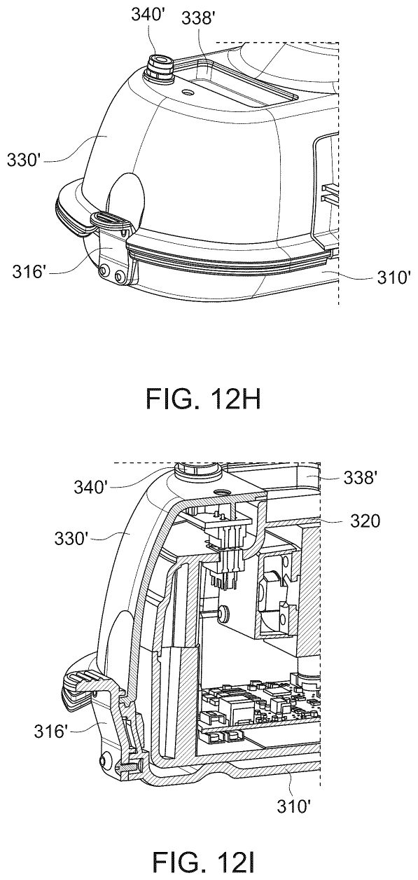

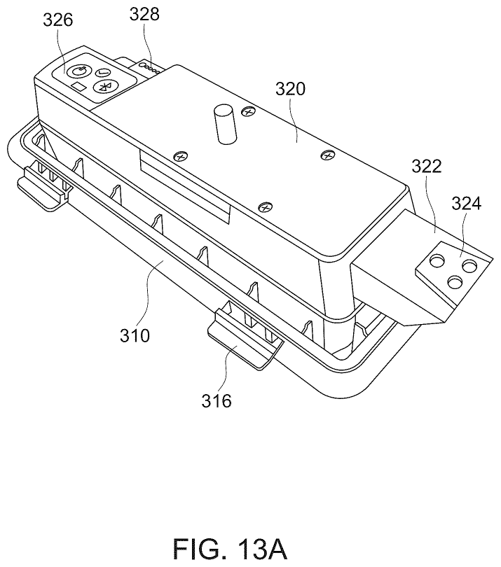

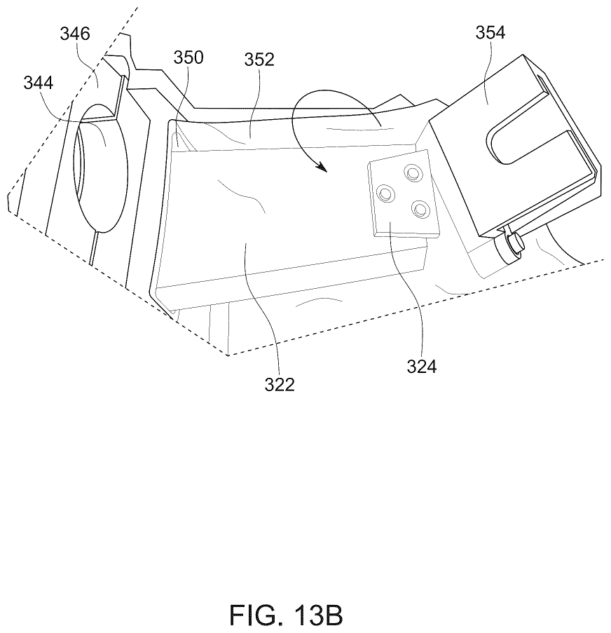

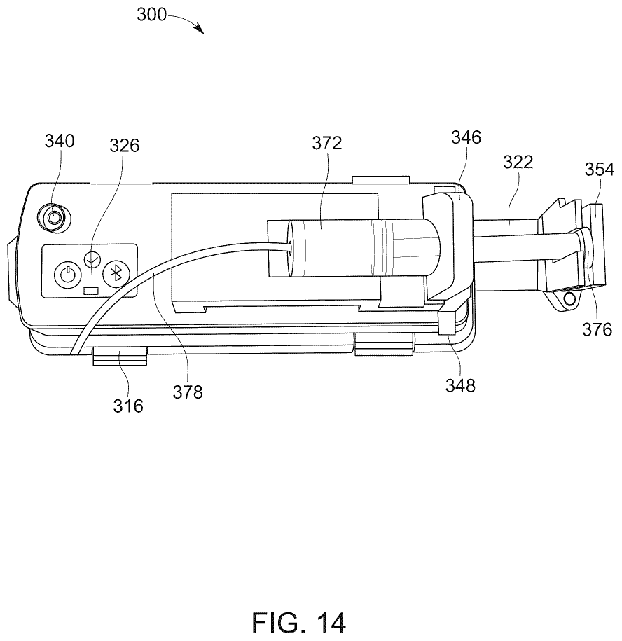

Although examples of a smart balloon inflation system 170 are described above and in the '097 Publication, other and/or additional features and/or components of a smart balloon inflation system are shown and described in connection with FIGS. 12A-14. One example of a smart balloon inflation system 300 is shown in FIG. 14. Example individual components of balloon inflation system 300 are shown and described in connection with FIGS. 12A-12D, while the balloon inflation system 300 is shown in various states of assembly in FIGS. 13A-13C. It should be understood that features described in connection with balloon inflation system 170 and/or the '097 Publication may be combined with or incorporated into balloon inflation system 300.

Referring now in addition to FIG. 12A, FIG. 12A is a perspective view of an example of a bottom enclosure 310 of a balloon inflation system 300. Generally, bottom enclosure 310 may include a flat bottom surface 312 and generally rectangular sidewall 314 extending upwardly from the bottom surface 312, the bottom surface 312 and sidewall 314 forming a recess to at least partially receive a motor or engine enclosure 320 snugly therein. In some examples, one or more latches 316 may be provided on the bottom enclosure 310. In this particular example, two latches 316 are provided on each long side of the bottom enclosure 310, each latch 316 being hingedly connected to the bottom enclosure 310 and configured to swing upwardly to engage a portion of the top enclosure 330 (described in greater detail below) to clamp the top enclosure 330 onto the bottom enclosure 310. It should be understood that different numbers and types of latches may be provided, and in some embodiments are provided on the top enclosure 330 instead of on the bottom enclosure 310. Because the balloon inflation system 300 may be used at the procedure table (e.g., in the operating room or cath lab), there typically must be a sterile barrier to separate any non-sterile objects from the sterile operating field. As is described in greater detail below, in some examples the motor or engine enclosure 320 is not cost-effective to provide as a disposable device. In such examples, the bottom enclosure 310 (as well as top enclosure 330 as described in greater detail below) may be provided as a sterile, disposable item intended for single-use only.

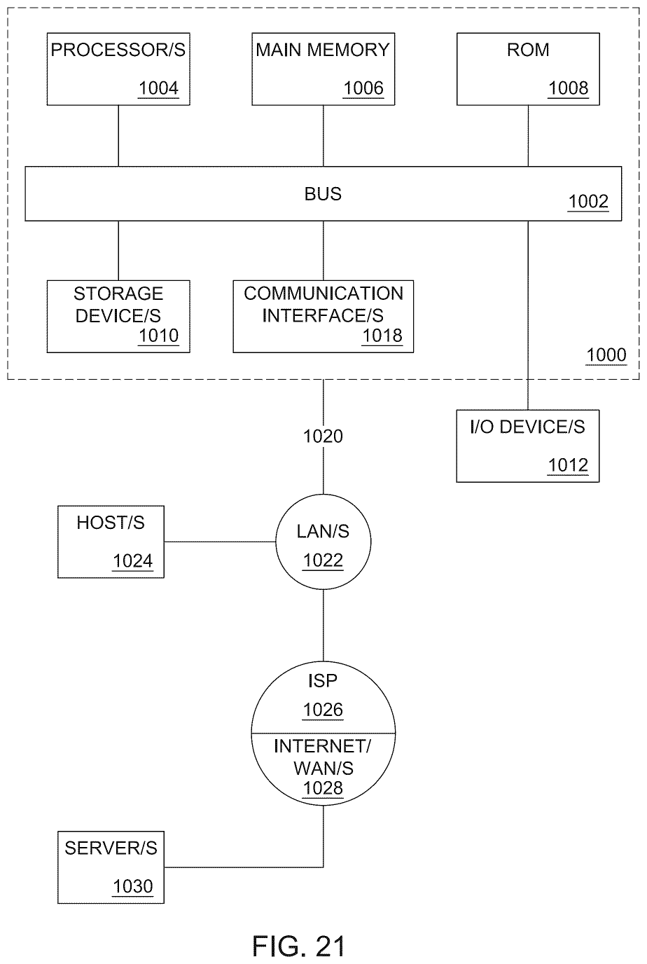

Referring now in addition to FIG. 12B, FIG. 12B is a perspective view of an example of a motor or engine enclosure 320 (which may be referred to simply as “motor” hereinafter) of the balloon inflation system 300. The motor may have a general size and shape so that a bottom portion thereof may be received within the bottom enclosure 310. As noted above, in some embodiments, it is preferrable for the motor 320 to be reusable. In some examples of use, the motor 320 is provided as a non-sterile component, but is partially or fully covered by a sterile enclosure (e.g., a single use sterile drape) during use. In some examples, the motor enclosure 320 includes various internal components, such as a motor, rechargeable battery, memory, and/or one or more processor(s), including example described below in connection with FIG. 21. The motor 320 in some examples may include a moving member 322 (which may be generally similar to moving member 180) at a rear portion thereof, the moving member 322 configured to telescope into or out of the motor enclosure 320 based on actuation of the motor within the motor enclosure 320. The motor within the motor enclosure 320 may be a stepper motor, a servo motor, a pneumatic motor, or any other suitable motor. In some examples, highly precise motor movement is controlled with integrated software and enabled by small steps in the motor construction. The moving member 322 in some examples includes a connector 324 fixed thereon, which may be used to couple a syringe plunger receiver 354 (described below) to the moving member 322.

In some embodiments, the motor 320 may include an indicator and/or input panel 326, for example near a forward end thereof. In the illustrated example, panel 326 may include a battery charge level indicator, a power button to turn the motor 320 on and off, a Bluetooth button to allow the motor 320 to wireless pair with a computer, tablet, or other device (including in some examples a component of delivery system 100 such as handle 110), and a status indicator (e.g., system ready for use, target inflation reached, general error occurred). The user interface in some examples can be accomplished with a membrane circuit with integrated lights and buttons or a digital display. In some examples, the system can be designed to restrict use if the battery level is too low to complete a procedure. Errors and other status indications can also be clearly shown using the connection to the external computer and/or tablet. Simple icons can be used on the panel 326 to notify the user as necessary. The motor 320 in some examples is battery powered and includes an internal, rechargeable battery that may be charged by docking the motor 320 on a docking station. In some examples, the motor 320 includes one or more connecting ports 328 near a front surface thereof, for example adjacent to the panel 326. The ports 328 may be used for charging, although in other examples separate ports may be used for charging the battery of motor 320. In some examples, the ports 328 may serve to connect the motor 320 to the delivery system (e.g., to handle 110) and/or to an external computer system. If motor 320 is provided as a reusable component, it is contemplated that a hospital, cath lab, or other site would have two or more of the motors 320 on-site which would allow for easy swapping out of one motor 320 for another, for example if a motor 320 had a low battery charge.

Referring now in addition to FIGS. 12C-12D, FIGS. 12C-12D are perspective views of the front and rear, respectively, of an example of a top enclosure 330 of a balloon inflation system 300. Generally, top enclosure 330 may include a bottom rectangular lip 332, a generally rectangular sidewall 334 extending upwardly from the lip 332, and a top surface 336. The side wall 334 and top surface 336 may form a recess to at least partially receive the top of motor 320 therein. In some examples, the one or more latches 316 of the bottom enclosure 310 may snap lock onto the lip 332. The front of the top surface 336 may include a window 338 which may generally match the size and shape of the panel 326 so that the panel 326 is visible and/or available for interaction through the window 338 when the balloon inflation system 300 is assembled. The top enclosure 330 may in some examples include a cable port 340, which in the illustrated embodiment is positioned adjacent to the window 338. In some examples, one or more cables may be plugged into the ports 328 of the motor 320 via cable port 340 to provide a wired connection (e.g., data connection) between the motor 320 and another component of the system. It should be understood that, although the term “window” is used in respect to window 338, the window 338 is not necessarily an opening, but may be a flexible, clear membrane that provides a sterile barrier while still allowing viewing and actuation of controls on the panel 326.

As best shown in FIG. 12D, a syringe dock may be positioned on top of the top surface 336 of the top enclosure 330. The syringe dock may include a distal cradle 342 which may be a generally semicircular recess sized and shaped to receive a barrel 372 of a syringe 370 (shown for example in FIG. 12E). The syringe dock may also include a proximal cradle 344 which may be generally semicircular, and a stabilizer 346 may be hingedly coupled to the proximal cradle 344. With this configuration, a proximal portion of the syringe (e.g., flange 374) may be partially received within the proximal cradle 344, and the stabilizer 346 may be swung closed over the proximal cradle 344, for example with a portion of flange 374 being received within a complementary slot of the stabilizer 346. Another portion of flange 374 may be received within a complementary slot of the proximal cradle 344. The stabilizer 346 may also include a lock 348 which may be engaged or actuated to lock the stabilizer 346 in the closed condition (e.g., as shown in FIG. 12D). The rear surface of the top enclosure 330 may include an opening 350 which may be generally rectangular, or otherwise sized and shaped to allow for moving member 322 to pass through as the moving member 322 telescopes into or out of the motor enclosure 320. In some examples, the top enclosure 330 includes a sterile pouch 352 coupled thereto, for example surrounding the opening 350. A syringe plunger holder 354 may be fixed to the sterile pouch 352, for example by having a top portion and a bottom portion clamped over the pouch 352. Structurally and functionally, the syringe plunger holder 354 may be similar to the proximal end of moving member 180 (e.g., as shown in FIGS. 9-10). In some examples, one or more handle holders 356 may be coupled to the top enclosure 330, the handle holders 356 configured to receive a handle of a delivery system (e.g., handle 110 of delivery system 100) to temporarily couple the handle to the balloon inflation system 300 for easy transport prior to use. As with bottom enclosure 310, top enclosure 330 (including sterile pouch 350 and syringe plunger holder 354 connected thereto) may be provided as a sterilized, single use component intended for disposal after use. In some examples, the openings between the top enclosure 330 and the bottom enclosure 310 may have a tongue-and-groove or similar interface so that neither a person nor a tool is able to contact the non-sterile motor enclosure 320 through the assembled top and bottom enclosures 330, 310.

Now referring in addition to FIG. 12E, FIG. 12E shows an example of a syringe 370 that may be used with balloon inflation system 300. Syringe may be a disposable, single-use syringe and include a barrel 372 for holding fluid (e.g., saline), a flange 374 which may be to assist in manually gripping the syringe 370 and/or locking a portion of syringe 370 to the top enclosure 310, and a plunger 376 which may be depressed to pressurize fluid within the syringe 370. A fluid line 378 may be coupled to a distal end of the barrel 372 to fluidly couple the syringe 370 to another device, such as a handle (e.g., handle 110) of a delivery system (e.g., delivery system 100). In some examples, the syringe 370 may be provided to the end user pre-attached to the delivery system.

FIGS. 12F-12I show alternate versions of the top enclosure 330′ and the bottom enclosure 310′. The top enclosure 330′ is generally similar to top enclosure 330, for example including a window 338′, a cable port 340′, an opening 350′ for receiving therethrough a moving member 322 of the motor 320, and similar or identical structures for receiving syringe 370. The bottom enclosure 310′ may have a similar structure and function as bottom enclosure 310, including to assemble with the top enclosure 330′ to house motor 320 therein. One difference between the top enclosure 330 and bottom enclosure 310 compared to top enclosure 330′ and bottom enclosure 310′ is a latching mechanism. For example, in the particular embodiment shown in FIGS. 12F-I, the forward or distal end of the bottom enclosure 310′ includes a latch 316′ that can snap to or otherwise engage a complementary locking surface of the top enclosure 330′, while the rear or proximal end of the bottom enclosure 310′ includes one or more (two shown in FIGS. 12F-G) tabs 317′ that can slidingly mate with a corresponding receiver 331′ of the top enclosure 330′. With this design, the user may hook the proximal end of the top enclosure 330′ into the proximal end of the bottom enclosure 310′ by hooking or sliding the tab(s) 317′ into the receiver 331′, and then push down the distal end of the top enclosure 330′ to engage the snap lock via latch 316′. However, it should be understood that the position of the latches and/or other mechanisms, as well as the number and type of such mechanisms, can be altered as desired without departing from the scope of the disclosure.

Now referring in addition to FIG. 13A, FIG. 13A shows a state of partial assembly of the balloon inflation system 300 in which the motor 320 has been placed into the bottom enclosure 310. In some examples, this first step may be performed by a non-sterile circulator (e.g. non-sterile nurse or other technician). The top enclosure 330 may be placed over the motor 320, and the latches 316 may be used to lock the top enclosure 330 to the bottom enclosure 310. This step may be performed by a sterile technician. During the assembly, as best shown in FIG. 13B, the moving member 322 of the motor 322 may be passed through the opening 350 of the top enclosure 330 and into the sterile pouch 352. At this point, the syringe plunger holder 354 may be moved (as indicated by the arrow in FIG. 13B) and then coupled to the connector 324, for example via a snap fit or screws. This step may also be performed by the sterile technician. With this configuration, the sterile barrier of pouch 352 is maintained while the syringe plunger holder 354 is fixedly coupled to the moving member 322.

During assembly, the plunger 376 of the syringe 370 may be positioned within the slot of the syringe plunger holder 354, the syringe flange 374 may be placed into the complementary slot of the proximal cradle, and the stabilizer 346 may be closed over the flange 374 and locked closed, with the barrel of 372 of the syringe received within the distal cradle 342.The coupling of the syringe 370 to the top enclosure 330 and to the syringe plunger holder 354 may also be performed by the sterile technician. The final assembled condition (prior to coupling the delivery system to the smart balloon inflation system 300) is depicted in the example of FIG. 14. At this point, any cables or wires from the delivery system may be plugged into the motor 320 via cable port 340, and the handle of the delivery system may be placed into the handle holders 356 (if included).

Although the example(s) of smart balloon inflation system 300 shown and described above is a highly portable element that can be placed within sterile field, in other embodiments, alternate designs of the smart balloon inflation system may be mounted on a cart or on a bed rail. With this type of configuration, the motor may be plugged directly into electrical mains supply instead of relying on a rechargeable internal battery. In these embodiments, the smart balloon inflation system would not need a specialized sterile enclosure as it may instead be draped with a standard surgical drape while in the sterile field. However, one potential disadvantages to this type of embodiment is that the syringe 370 would not be accessible within the sterile field, so that if manual operation of the syringe 370 becomes desired or necessary, a non-sterile clinician may need to be available.

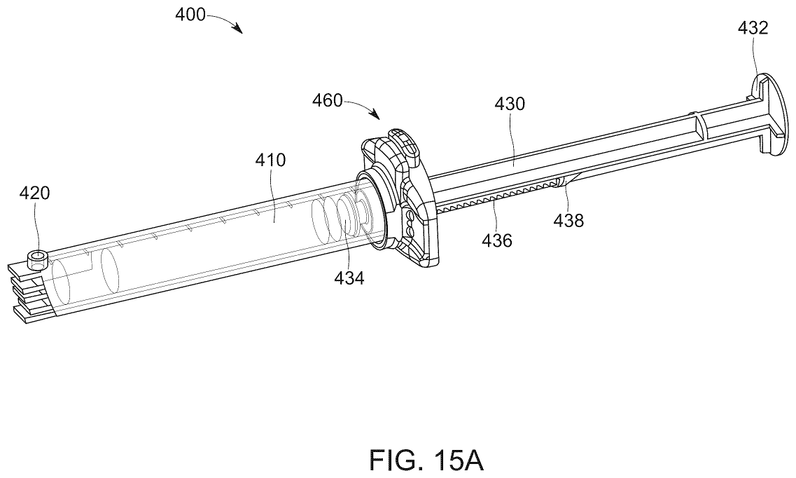

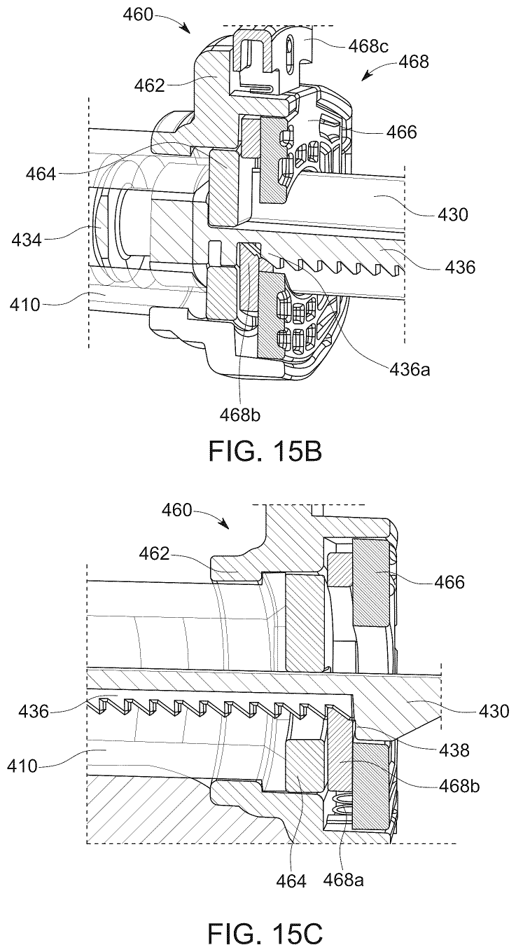

Now referring in addition to FIG. 15A, FIG. 15A is a perspective view of an example of a syringe 400 that may be used with balloon inflation system 170 or 300 (e.g., in place of syringe 174 or 370). In the illustrated example, syringe 400 includes a barrel 410 for holding fluid (e.g., saline) and an outlet port 420 at or near a distal end of the barrel 410. The outlet port 420 may couple to fluid tubing (e.g., fluid 378) which may in turn be provided pre-assembled to a delivery system (e.g., to handle 110 of delivery system 100). In some embodiments, the barrel 410 may include markings or other indicators that indicate fluid volume. In some examples, markings may be included for achieved valve size (e.g., in either diameter or area) for use during manual inflation. A plunger 430 may include a proximal cap 432 (e.g., thumb rest) and a distal seal 434 that sealingly engages an internal surface of the barrel 410. As with most typical syringes, pressing the plunger 432 into the barrel 410 forces fluid out of the syringe 400, e.g. via outlet port 420. In the specific example shown in FIG. 15A, the syringe 400 is a 45 ml syringe, and may be compatible with all offered sizes of the prosthetic heart valve (e.g., multiple size options of prosthetic heart valve 10) as well as all offered sizes of the delivery system (e.g., a small and large version of delivery system 100).

Whether syringe 400 is used with balloon inflation system 300 (or balloon inflation system 170) within a sterile field, or with a bed-or rail-mounted balloon inflation system outside the sterile field, it may be desirable for the syringe 400 to be capable of decoupling from the balloon inflation system 300 so that a prosthetic heart valve deployment may be performed manually with syringe 400. This may be desirable for example if there is a power interruption (e.g., to the balloon inflation system if it is coupled to electrical mains, or a low or dead battery or power transmission problem with a rechargeable battery), or other mechanical or electrical issue that prevents the balloon inflation system 300 from providing the desired automated (or semi-automated) operation of syringe 400 to inflate the balloon (e.g., balloon 136) of the delivery system (e.g., delivery system 100).

In one example of a malfunction of balloon inflation system 300, the motor 320 becomes incapable of continuing to advance plunger 376 after the balloon (e.g., balloon 136) has already begun to inflate and expand the prosthetic heart valve (e.g., valve 10). In this scenario the relatively high pressure within the balloon could force fluid back into the syringe 370, causing the balloon to at least partially deflate, which could result in the prosthetic heart valve fully decoupling from the balloon prior to being secured within the native valve annulus. In the example of syringe 400 shown in FIGS. 15A-15C, a mechanism is provided to prevent the plunger 430 from being forcibly retracted as a result of pressure within barrel 410 (which could be caused by high pressures within the balloon of the delivery system). For example, FIGS. 15B-15C illustrate enlarged cut-away views of a flange 460 of the syringe 400. It should be understood that flange 460 may interact with components of balloon inflation system 300, such as by being receive within complementary slots of proximal cradle 344 and/or stabilizer 346, in a similar manner as described in connection with syringe 370.

Referring now in addition to FIGS. 15B-15C, FIGS. 15B-15C illustrate that plunger 430 may include a ratcheting mechanism that interacts with components of flange 460 such that, once the plunger 430 has begun advancing (e.g., manually by a user) distally into barrel 410, it is not capable of retracting without active intervention by a user. For example, plunger 430 may include a plurality of ratchet teeth 436 extending therefrom, each ratchet tooth 436 having a ramped surface and a flat surface opposite the ramped surface. The flange 460 may include a housing 462, a distal end of which may be coupled to a proximal end of barrel 410. The housing 462 may partially or completely house a distal plate 464, a proximal plate 466, and an actuator 468 positioned between the distal plate 464 and proximal plate 466. Each plate 464, 466 and the actuator 468 may include a through bore or other opening to allow for passage of the plunger 430 through the structure. In some examples, distal plate 464 may help to prevent the plunger 430 from rotating relative to the barrel 410. This functionality may be achieved by flanking the shaft of the plunger 430, and distal plate 464 may have a square shape that fits in a pocket of housing 462. In some examples, the proximal plate 466 may attach to the flange 462 via screws, welding, gluing or another suitable mechanism, which may help to keep all the internal components together and keep the plunger 430 from pulling completely out during deflation.

While distal plate 464 and proximal plate 466 are preferably static or fixed relative to the housing 462, actuator 468 may be moveable relative to the housing 462. For example, the actuator 468 may include a biasing member 468a (such as a spring), a tooth 468b, and a button 468c. As the plunger 430 is advanced distally into barrel 410, the ramped surface of a plunger tooth 436 advances against the ramped surface of the actuator tooth 468b. Because the plunger 430 is substantially only capable of distal and proximal movement, the contact between plunger tooth 436 and actuator tooth 468b during advancement forces the actuator 468 to move downward while compressing spring 468a. Once the ramped surfaces of the teeth 436, 468b clear each other, the spring 468a is able to decompress, forcing the actuator tooth 468b back up so that the ramped surface of the actuator tooth 468b contacts the ramped surface of the next plunger tooth 436. With this example ratcheting mechanism, if the motorized system that is advancing the plunger 430 to inflate the balloon of a delivery device fails mid-inflation, and the user detaches the syringe 400 from the motorized system, the balloon will not be able to depressurize and force the plunger 430 to retract, since contact between the flat surfaces of the actuator tooth 468b and a particular plunger tooth 436 will prevent such movement (see, e.g., contact between actuator tooth 468b and corresponding plunger tooth 436 shown in FIG. 15C). Notably, if the motorized system fails mid-inflation, and the user does not detach the syringe 400 from the motorized system, the balloon is still not able to depressurize because the motor provides enough holding force, even during a power failure, to keep the syringe 400 from back driving enough to cause valve embolization. In other words, the ratcheting mechanism provides for one-way movement of the plunger 430 distally into barrel 410, and not reverse movement absent intervention by a user. If a user does want to intervene and disengage the ratcheting mechanism, the user may press the button 468c (which may be referred to as a “release button”) downward to compress spring 468a, allowing the plunger 430 to be retracted if desired. In some examples, contact between the actuator 468 and interior surfaces of the flange housing 462 limit the total distance which the actuator 468 is capable of moving relative to the flange housing 462. If a mechanism like actuator 468 is provided, it should be understood that other modifications may be made to balloon inflation system 300 (or similar systems). For example, if a stabilizer 346 is included, the stabilizer 346 may include an opening, recess, or other feature to ensure that the release button 468c is not maintained in the depressed condition while the syringe 400 is engaged with the balloon inflation system 300. However, in some examples, the stabilizer 346 may be configured to keep actuator 468 depressed, which may allow the syringe plunger to move freely while the syringe is docked in the top enclosure 330. In some examples, the primary failure mode that syringe 400 may mitigate is engine failure in the form of locking up or losing power. If the motor uses a stepper or servo motor, for example, these failure modes would likely result in the plunger being frozen in place, and not moving or losing pressure. So in this case, upon failure, the operator may quickly unlock the stabilizer 346, thereby allowing the button 468 to pop up to allow the ratcheting mechanisms to engage, after which removal of the syringe 400 and manual use by the operator could be performed as described herein.

Still referring to FIGS. 15A-15C, whether or not inflation is completed using the automated (or semi-automated) balloon inflation system 300 (or a similar balloon inflation system), it may be desirable to use syringe 400 to manually deflate the balloon at or near the end of the procedure, including in the case where a malfunction of the balloon inflation system 300 occurs. When deflating a balloon (e.g., balloon 136) following deployment of a balloon-expandable heart valve (e.g., valve 10), it may be desirable to deflate the balloon very rapidly to minimize the time that an inflated balloon is potentially blocking blood flow within the heart. If balloon inflation system 300 is working properly, the balloon inflation system 300 may be able to rapidly withdraw the plunger 430 to deflate the balloon as fast as desired. However, if syringe 400 is being used to manually deflate the balloon (due to either preference or necessity), and a user manually withdraws plunger 430 rapidly, the plunger 430 may tend to want to get “sucked forward” if tension on the plunger 430 is released immediately after rapidly retracting it. In other words, if a user manually pulled plunger 430 to its fully retracted position quickly, and did not maintain force on the plunger 430 to hold it in that position, the plunger may pull forward and slow the deflation of the balloon. In some examples, in order to prevent this from happening, the ratchet teeth 436 of the plunger may be configured to automatically lock the plunger 430 from forward movement when it is in the proximal-most, fully retracted position. For example, referring to FIG. 15B, the distal-most ratchet tooth 436a of the plunger 430 may include (i) a proximal flat surface generally similar or identical to the proximal flat surfaces of the other ratchet teeth 436, (ii) a ramped surface generally similar or identical to the ramped surfaces of the other ratchet teeth 436, but (iii) a distal flat surface that has a length to engage a proximal flat surface of actuator tooth 468b positioned beneath the ramped surface of the actuator tooth 468b. With this configuration, when the plunger 430 is in the proximal-most and/or fully retracted position, the distal-most ratchet tooth 436a of the plunger has a distal flat surface in contact with a proximal flat surface of actuator tooth 468b, preventing distal movement of the plunger 430 until a user depresses release button 468c. Thus, if the user manually retracts the plunger 430 rapidly, once the distal-most tooth 436a of the plunger 430 engages the actuator tooth 468b while the button 468c is released, the plunger 430 will lock in that fully retracted position, preventing the tendency of the plunger 430 to pull forward immediately after the rapid retraction. If the plunger 430 needs to be pressed forward, the lock may simply be disengaged by depressing the button 468c.

In an emergency bailout situation in which the user needs (or wants) to manually use syringe 400, it may also be useful to have a feature to assist the user in delivering the correct volume of fluid from the barrel 410 into the fluid line leading to the balloon. If balloon inflation system 300 is used successfully, the fluid may be delivered from the syringe 400 into the fluid line toward the balloon in a highly precise manner. However, in the absence of the balloon inflation system 300, precision in amount of fluid delivered may be harder to achieve if syringe 400 is operated manually. The syringe 400 is typically always provided to the user completely filled, which may create a possibility of over-filling of the balloon if the syringe 400 is used manually. In some examples, the syringe 400 may be provided with a plunger 430 that is adapted for use with a particular size prosthetic heart valve. For example, if prosthetic heart valve 10 is provided in four different sizes, four different plungers 430 may be available for use with syringe 400, with a particular plunger being provided with the syringe 400 depending on the size of the prosthetic heart valve 10 being used in the procedure. Each plunger may include stop 438, the positioning of the stop 438 along the plunger 430 being different depending on the size of the prosthetic heart valve 10. For example, as shown in FIG. 15C, the plunger 430 may include a stop 438 that includes a large relatively flat distal surface that is unable to move past actuator tooth 468b, regardless of the position that the actuator tooth 468b is in (e.g. whether the spring 468a is fully compressed or fully extended). The stop 438 may be positioned along the length of the plunger so that the stop 438 engages the actuator tooth 468b and prevents further advancement of the plunger 430 once a maximum desired volume has been passed into the balloon via the syringe 400. In other words, the stop 438 may prevent overfilling of the balloon, and the total volume of fluid that has been delivered when the stop 438 is engaged may depend on the position of the stop 438 along the plunger 430, and may correspond to the maximum desired volume of balloon inflation for the particular size prosthetic heart valve 10 being implanted. Further, in some examples, the barrel 410 may include markings or other indicia that indicate the minimum and maximum inflation volumes of a balloon for a particular size prosthetic heart valve, so that the user, if using the syringe 400 manually, may be able to stop filling the balloon at a smaller total volume prior to the stop 438 engaging the actuator tooth 468b.

As should become clear, systems and method described herein may be useful for various stages of a prosthetic heart valve implantation, including preparation stages, deployment stages, and post-dilatation stages. Features relating to the deployment phase are described below (in addition to features already described above), and that description is followed by descriptions of post-dilatation and de-airing features. Although deployment phase features (and/or methods) are described in connection with balloon inflation system 300, it should be understood that these features (and/or methods) may apply to other balloon inflation systems, such as balloon inflation system 170, and various types of syringes, including syringes 174, 370, or 400.

Referring now in addition to FIG. 16A, FIG. 16A is a schematic view of balloon inflation system 300 with related components. In the illustrated example, the system includes a balloon inflation system 300 including syringe 370 mounted thereon (although syringe 400 may be used instead). In some examples, the balloon inflation system 300 may be operatively coupled to a balloon catheter handle 110 (e.g., via fluid line 378 and cables that transmit power and/or data). As described above, the balloon catheter handle 110 may include balloon actuator 120, steering knob 112, commissure alignment actuator 114 and/or axial alignment actuator 116. The balloon catheter handle 110 may be operatively coupled to inflatable balloon 136. A sensor 137, which in some examples is a pressure sensor, may be positioned anywhere within the path of the inflation lumen that extends between syringe 370 and the interior of the balloon 136. In the illustrated example, the pressure sensor 137 is mounted to an internal shaft within the balloon 136, but it should be understood that this is only one exemplary position. For example, in some embodiments, pressure sensor 137 may be provided within handle 110. The pressure sensor 137 may be operatively coupled to the balloon inflation system 300 so that data (e.g., pressure readings) may be transmitted from the pressure sensor 137 to the balloon inflation system 300. Balloon inflation system 300 may be operably coupled to balloon catheter handle 110 and may in some examples also be operably coupled to a computer 560 (which may have an integrated display) and/or to a mobile display 570, such as a tablet. Computer 560 and tablet 570 may in some examples individually, in combination, or along with other components, form a computer system (or a portion thereof) as described in connection with FIG. 21. The data connections between the balloon inflation system 300 and the balloon catheter handle 110 and/or computer 560 may be wired or wireless. In some examples, the computer 560 may receive real-time readings from pressure sensor 137 (which may be relayed through inflation system 300), and those real-time readings may be graphically displayed on the computer 560 and/or on an associated tablet 570. Similarly, in some examples, data regarding the state of inflation of balloon 136, including for example volume of inflation media passed into the balloon 136, may be displayed on the computer 560 and/or on an associated tablet 570. For example, due at least in part to the motorized driving of syringe 370 by inflation system 300, the inflation system 300 may know at any point how much inflation media has been passed from the syringe 370 to the balloon 136 (or vice versa), which information may be transmitted to the computer 560 for display along with the pressure data.

FIG. 16B illustrates an example of a balloon compliance curve for balloon 136 illustrating the relationship between balloon pressure and balloon volume. The solid line represents the baseline or “open air” pressure-volume curve 510 when the balloon 136 is inflated without coming into contact with other structures. However, once the balloon 136 makes contact with a surface, such as the aortic valve annulus, the pressure-volume curve shifts from the baseline, shown in the dashed line 520. Curves 510, 520 are identical prior to the balloon 136 inflating into contact with the native aortic annulus. However, upon contact, the pressure-volume curve shifts from baseline 510 by an amount, with this change or delta represented by arrow 530. This delta 530 is the result of the native tissue compliance applying a compressive force against the expanded balloon. This information may be utilized to help determine when the prosthetic heart valve 10 has been expanded the desired amount. For example, a particular amount of deviation 530 between the baseline pressure-volume curve 510 and the actual pressure-volume curve 520 during implantation may be determined as the amount of deviation 530 that corresponds to optimal prosthetic heart valve 10 expansion within the aortic valve annulus. This value of desired deviation 530 may be determined, for example, via testing across multiple patients or by analysis of data of a number of actual implantations. As noted above, the volume of fluid passing from syringe 370 into balloon 136 may be tracked in real time, and the pressure may also be tracked in real time via one or more of the pressure sensors 137 described above. Thus, during a valve implantation using prosthetic heart valve 10 and balloon inflation system 300, the real time pressure-volume curve 520 may be displayed (e.g., on computer 560 and/or tablet 570), along with the expected baseline press-volume curve 510, for reference by the user, allowing the user to use the displayed data to confirm the desirability of the procedure or otherwise to alter the procedure based on the data.

Referring again to FIG. 16A, fluid may pass in either direction between the balloon inflation system 300 and the handle 110 of the delivery system, and the instruction signals for activating the balloon inflation system 300 may pass from the handle 110 to the balloon inflation system 300. The physician or other operator may manually control the delivery system, including using handle 110 to implant the prosthetic heart valve 10 into the patient, including via controlling the inflation and deflation of the balloon 136 via the actuator 120. During the procedure, data obtained from the procedure, such as volume that has passed from the balloon inflation system 300 toward the balloon 136 (or vice versa), the current area of the balloon 136 (which may be calculated based on the fluid volume), and the current pressure within the system, may all be transmitted to and/or displayed on the computer 560 and/or tablet 570. As noted above, the data transmission may be via a wired or wireless connection. The physician may view the display(s) 560, 570, which may include the above-noted data, as well as other information such as fluoroscopic images of the patient's anatomy. Other support personnel may similarly view the data on the display(s) 560, 570, and either the physician or the support personnel may input parameters, such as a target volume for the inflation of balloon 136 via the computer(s) 560 and/or display(s) 570. The computer(s) 560 and/or display(s) 570, in turn, may communicate such parameters to the balloon inflation system 300, for example by setting the target volume such that the balloon inflation system 300 does not inflate the balloon 136 beyond the target volume, unless either a user overrides the balloon inflation system 300.

FIG. 16C illustrates an exemplary screen that may be shown on the computer 560 and/or tablet 570 as part of a planning stage prior to implanting prosthetic heart valve 10 into the patient. In this exemplary screen, one or more inputs may be entered for use during the procedure. One exemplary input is the target annulus area, which represents the size of the patient's native valve annulus. In this particular example, the value entered is 623 mm². Another exemplary input is the desired oversizing percentage of the prosthetic heart valve 10. In other words, it is often desirable to target an area/size for the prosthetic heart valve 10 that is larger than the area/size of the patient's native valve annulus, for example to create enough friction to help maintain the prosthetic heart valve 10 in place during normal operation of the prosthetic heart valve 10. In this particular example, the value entered for the percent oversizing is 5.3%. It should be understood that all numbers and values provided with respect to FIGS. 16C and 16D are merely exemplary and are not intended to be limiting, but rather illustrate one example of inputs and outputs to better illustrate the related concepts. Based on the input during the planning stages, certain outputs may be provided for review and confirmation by the physician and/or support personnel, such as the target area for the prosthetic heart valve 10, which may be calculated by applying the oversizing percentage to the patient's annulus area. A particular size prosthetic heart valve 10 may be suggested by the output as well. For example, prosthetic heart valves 10 are typically provided in a different selection of sizes, and the physician will choose the appropriate size selection for the particular patient. In this example, a prosthetic heart valve 10 size of 29 mm is recommended based on the inputs. Another output that may be provided to the users is a suggested total inflation volume that should be pushed from the balloon inflation system 300 to achieve the desired expansion size of the prosthetic heart valve 10. In this particular example, a total inflation volume of 33 mL is suggested. The suggested inflation volume may be provided based on, for example, pre-determined correlations derived from testing that relate inflation volume to valve area upon expansion of the balloon 136. Still another output that may be provided is a target pressure for the balloon 136 to achieve the desired expansion size of the prosthetic heart valve 10. In this particular example, a target pressure is provided as 6.3 atm. It should be understood that, although various recommended values are output based on the input data, the physician has the control to override the recommended values based on his or her experience.

FIG. 16D illustrates an exemplary screen that may be shown on the computer(s) 560 and/or tablet(s) 570 as part of the mid-procedure stage of implanting the prosthetic heart valve 10 into the patient following the planning stage shown in FIG. 16C. The patient's annulus area and selected size of the prosthetic heart valve 10 from the planning stage of FIG. 16C may be displayed on the mid-procedure screen of FIG. 16D, as well as a current status of the procedure, for example “inflating” or “deflating.” The mid-procedure screen of FIG. 16D may show a plurality of sections that provide a current procedure parameter versus the target procedure parameter in order to assist the user(s) in understanding the progress of the deployment of the prosthetic heart valve 10. For example, a valve area section may provide the target prosthetic heart valve 10 expansion size/area compared to the current prosthetic heart valve 10 expansion size/area during inflation of the balloon 136. The screen illustrated on tablet 570 shows the previously chosen target size of 656 mm² compared to the current mid-inflation size of the prosthetic heart valve 10 of 326 mm². In addition to providing the values, a graph, such as a progress bar, may be shown illustrating the current expansion size as a percent of the patient's valve annulus size compared to the desired expansion size as a percent of the patient's valve annulus size. The target value from the planning stage, which in this example is a 5.4% oversizing, is indicated on the progress bar (e.g. 105.4% of the patient's annulus size), along with the current status (e.g., a mid-procedure size of the prosthetic heart valve PHV of 56.0% of the patient's annulus size). Similar information panels with progress bars may be provided for other parameters, such as a current balloon pressure (e.g., of 6.0 atm) versus the planned target balloon pressure (e.g., of 6.4 atm). Another information panel for current inflation volume (e.g., 27.2 mL at the illustrated stage of the procedure) versus the target inflation volume (e.g., 33.2 mL from the planning stage) may be provided along with a graphical representation, for example via a progress bar that includes an indicator of the target inflation volume.

FIG. 16D also illustrates a graph that plots the pressure within the balloon 136 versus the area of the balloon 136 as the procedure continues. Similar to the compliance curve in FIG. 16B, a baseline pressure-area curve 510 may be provided as a static, known relation that would be expected for inflating the balloon 136 in “open air.” As the balloon 136 inflates, the actual procedural pressure-area curve 520 may be plotted as the pressure is detected while the area is either sensed (e.g. using a strain gauge on the balloon) or computed (e.g., based on known correlations between fluid volume and area for the balloon 136. As shown in FIG. 16D, a deviation 530 between the procedural pressure-area curve 520 from the baseline pressure-area curve 510 results. It should be understood that the large deviation 530 shown in FIG. 16D is merely for illustrative purposes, and the deviation 530 of the size shown is not intended to represent an actual expected level of deviation. Regardless, the deviation 530 between the baseline curve 510 and the procedural curve 520 may provide important information to the users that may be utilized to confirm that the procedure is proceeding as intended, or otherwise that a potential problem has occurred that needs to be investigated and/or addressed. For example, as noted above, a known target deviation 530 between the baseline curve 510 and the mid-procedure curve 520 may be either set or understood to be a deviation that is desired. If the illustrated deviation 530 is near or equal to the target deviation 530, and the other target parameters (e.g., prosthetic heart valve size, balloon pressure, and/or inflation volume) are all at or near their target values, the physician may determine that the procedure has met all of the targets and that the prosthetic heart valve 10 has been appropriately deployed. It should be understood that the physician may use other information, including fluoroscopic images and his or her general experience and knowledge, to aid in this determination. However, if the procedural pressure-area curve 520 is deviating from the baseline curve 510 significantly more than expected, such a deviation 530 may be indicative of a potential problem. For example, in the illustrated example of FIG. 16D, the pressure of the balloon 136 has increased much sooner than expected, meaning that despite the prosthetic heart valve 10 having been only partially expanded toward the target size, the balloon 136 and prosthetic heart valve 10 are experiencing significantly higher forces than expected, which may be the result of the native tissue pressing against the prosthetic heart valve 10. Reasons for this deviation may include, for example, an incorrectly measured size of the patient's valve annulus and/or significantly greater calcification of the native valve than expected. If the physician continued inflating the balloon 136 despite the large deviation 530 shown, the annulus and/or balloon 136 may be at risk of rupture. Thus, the physician may pause the inflation of the balloon 136 using the actuator 120 on the handle 110, and assess the situation to determine the cause of the deviation 530, and may adjust the procedure accordingly. In some embodiments, the physician or support personnel may interact with the computer(s) 560 and/or display(s) 570 to update the target parameters based on information learned from the assessment following pausing of inflation. In some embodiments, the physician or support personnel may look to the live fluoroscopic images for valve expansion and if it is believed that the prosthetic heart valve 10 is anchored, the balloon may be deflated for further investigation using, for example, fluoroscopy and contrast injections. It should be understood that, although the term “pressure-area curve” is used herein, the “area” portion of the curve may be replaced with any parameter relating to the size of the implant, such as diameter (e.g., a “pressure-diameter curve”) or volume of inflation media (e.g., a “pressure-volume curve”), with similar results.

In some embodiments, the computer(s) 560 and/or tablet(s) 570 may be programmed to provide alerts to the users when parameters are approaching, at, and/or exceeding the target parameters. Such alerts may be purely information, or may otherwise cause a procedural change. For example, when the sensed pressure of the balloon 136 achieves the target pressure (or otherwise exceeds the target pressure, or exceeds the target pressure by a pre-determined buffer value, for example 5%), the computer(s) 560 and/or tablet(s) 570 may create a purely informational alert that the target pressure has been reached or exceeded (either by any amount or the predetermined buffer amount), or may create an alert that also signals the balloon inflation system 300 to stop inflating the balloon 136. If the alert causes such an action, the users will be able to override that alert by dismissing it, for example via interaction with the computer(s) 560 and/or tablet(s) 570, and then continue inflating the balloon 136 if deemed appropriate to do so. Other similar types of alerts for other target values may be similarly provided.

Currently used methods for balloon-expandable prosthetic heart valve implantation have no control over fine-tuning the rate of inflation of the balloon of the delivery device (and thus the rate of expansion of the prosthetic heart valve mounted thereon). Rather, with manual syringes being the current standard, the rate of balloon inflation is entirely dependent on the user's push force on the syringe plunger. With a motorized inflation device, such as balloon inflation system 300, the rate of inflation can be precisely and accurately manipulated. There are a number of reasons that it may be desirable to set a particular rate of inflation of the balloon during deployment of the prosthetic heart valve. For example, patients that are particularly sick may not be able to tolerate rapid pacing of the heart (which is a standard procedural step in most transcatheter aortic valve replacement procedures) for any extended amount of time. Thus, for these patients, a user may prefer relatively fast rates of inflation to minimize the amount of time that rapid pacing may be necessary. However, in other situations, it may be beneficial to perform balloon inflation slowly, for example because deployment of the prosthetic heart valve may be more predictable and it may be easier to react to changing conditions. As one example, the majority of foreshortening of the prosthetic heart valve typically occurs at some point within the first 20% of valve deployment, including between about 5% and about 15% of valve deployment, including about 10% of valve deployment. As used herein, references to percentages of valve deployments may be references to either diameter or total amount of fluid volume passed into the balloon. In other words, if 100 units of fluid need to be passed into the balloon to achieve full deployment, 20% of valve deployment may be achieved when 20 units of fluid are passed into the balloon. Because there are a relatively large number of changes happening in this early stage, it may be preferable for the balloon to inflate at a relatively slow rate so that the changes may become more predictable and so that it may be easier to identify if any adjustments are needed prior to completing the remaining 80%-95% of deployment. For example, the prosthetic heart valve may tend to rotate during this initial balloon expansion, and if commissure alignment is desired, the prosthetic heart valve may need to be re-aligned to compensate for this initial rotation. On the opposite side, when the balloon inflation is nearly complete with the prosthetic heart valve nearly fully deployed, for example when the prosthetic heart valve starts to contact the patient's tissue (e.g., the native valve annulus or native leaflets), it may be desirable for the balloon to inflate at a relatively slow rate to allow the tissue to adjust to the applied force. For example, patients receiving a balloon-expandable prosthetic heart valve typically have calcium deposits within the tissue of the native valve annulus. As the prosthetic heart valve contacts and exerts force against these calcium deposits, it may be preferably for the valve deployment to be relatively slow to allow the calcium to shift without piercing into the vasculature. However, between that first stage (e.g., about the first 10% of expansion) and the near-final stage (e.g., when the prosthetic heart valve contacts tissue), it may be preferable for the balloon to inflate at a very rapid rate since this middle portion of the inflation may be non-critical in terms of accurate placement, and a rapid rate of inflation during this middle portion can significantly reduce the overall time required for the prosthetic heart valve to be deployed via balloon inflation. In view of these factors, it would be preferable to achieve a staged inflation/deployment speed profile.

Now referring in addition to FIG. 16E, FIG. 16E illustrates a screen that may be displayed on tablet 570 providing for pre-programmed inflation speed profiles. For example, the computer 560 and/or tablet 570 and/or balloon inflation system 300 may be pre-programmed with different inflation rate options. In the exemplary configuration screen shown in FIG. 16E, three pre-programmed inflation rate profiles are presented, including a slow, medium, and fast profile. Each profile may include a relatively slow inflation rate for the initial stage of balloon inflation (e.g., first 5%, first 10%, first 15%, or first 20% of inflation). Each profile may include a relatively fast inflation rate for the second stage of balloon inflation (e.g., starting at about 5% to about 20% of inflation and ending at between about 70% and about 90% of inflation). Each profile may include a relatively slow inflation rate for the third stage of balloon inflation (e.g., starting at between about 70% to about 90% inflation and ending at 100% inflation). It should be understood that the first and third stages do not need to be the same inflation rate, but preferably both the first and third inflation rates are slower than the second inflation rate. In the illustrated screen of FIG. 16E, although relative differences in inflation rates between the different stages may be maintained, the total time to reach 100% inflation may be greatest in the pre-programmed “slow” option and smallest in the pre-programmed “fast” option. Although three examples are provided, it should be understood that any particular inflation rate profile may be programmed according to the user's desires, not just ones that may be pre-populated for the user. And although three stages of inflation are generally described, it should be understood that more or fewer stages may be used instead. Once the profile is selected, the settings may be downloaded or otherwise communicated to the balloon inflation system 300 (e.g., to the motor 320). Then, during use, the user may be able to simply press balloon actuator 120 a single time (or hold the button down) and the speed of inflation will change according to the selected profile. In this type of example, the balloon actuator 120 does not control the speed of inflation of the balloon 136, but rather just causes inflation or deflation when activated, with inflation/deflation being paused while the balloon actuator 120 is not activated. Rather, the motor 320 controls the inflation rate depending on the profile selected and the current stage of inflation. In some embodiments, the third stage of inflation may be a pre-set range, for example between 90% and 100% of balloon inflation. In other embodiments, the third stage of inflation may be based on detection of the system that the prosthetic heart valve 10 has made initial contact with the patient's anatomy, which may be determined by monitoring pressure and detecting a pressure spike that may result from the initial contact. It should be noted that, in the example screen shown on table 570 in FIG. 16E, options for pairing the balloon inflation system 300 (which may be referred to as an indeflator) to the tablet 570 and sending the selected speed profile back to the balloon inflation system 300 are shown as selectable options.

Although one option for achieving stages inflation at different inflation rates is via selecting a programmed inflation speed profile, in other examples the delivery system (e.g., delivery system 100) may be provided with features that allow for a user to actively adjust the rate of inflation of the balloon. For example, referring now in addition to FIG. 16F, FIG. 16F illustrates a handle 110′ of a delivery system that may be similar or identical to delivery system 100. Handle 110′ may be similar to handle 110 with one main exception. Handle 110′ includes a balloon actuator 120′ that is not an on/off (e.g., inflation, deflation, or neither) switch, but rather an adjustable inflation speed switch. For example, the user may press the balloon actuator 120′ forward to cause inflation of the balloon (e.g., balloon 136) or press the balloon actuator 120′ backward to cause deflation of the balloon. Instead of merely being on/off, the force applied to the balloon actuator 120′ may correlate with inflation rate, such that pressing the balloon actuator 120′ with more force (or so that balloon actuator 120′ has a greater travel distance) will cause faster inflation (or faster deflation), allowing the user to control the inflation rate with the balloon actuator 120′. Although balloon actuator 120′ is shown as a sliding switch, various other types of actuators with adjustable speed may instead be used, such as a dial or a trigger/push button. An alternate example for achieving variable rates of inflation as shown in FIG. 16G. Now referring in addition to FIG. 16G, FIG. 16G illustrates a handle 110″ of a delivery system that may be similar or identical to delivery system 100. Handle 110″ may be similar to handle 110 with one main exception. While handle 110″ includes a balloon actuator 120″ that is an on/off (e.g., inflation, deflation, or neither) switch, it also includes a secondary actuator 121″ which may be used to set and/or increase the inflation speed. In one example, actuator 120″ has a single base inflation rate, and if secondary actuator 121″ is depressed while actuator 120″ is actuated, the speed of inflation (or deflation) temporarily increases while the secondary actuator 121″ is depressed. In other examples, secondary actuator 121″ may be toggled between a high and low speed setting, and when the balloon actuator 120″ is actuated, inflation (or deflation) will proceed at a single speed, with the speed being dependent on whether the secondary actuator 121″ is toggled to the high speed or low speed. In this example, although secondary actuator 121″ is described as having a high or low speed setting, it should be understood that secondary actuator 121″ may toggle between more than two speed settings (e.g., slow, medium, fast).

In some prosthetic heart valve implantation procedures, after the prosthetic heart valve (e.g., prosthetic heart valve 10) is deployed into the native valve annulus, the prosthetic heart valve may need to be expanded further is a second balloon inflation procedure, for example to reduce or eliminate PV leak and/or to achieve better hemodynamics. This secondary expansion step may be referred to as “post dilatation.” In current procedures, to perform a post dilatation procedure, the prosthetic heart valve delivery system is removed from the patient, and a balloon aortic valvuloplasty (“BAV”) catheter is introduced into the vasculature and expanded into the previously-implanted prosthetic heart valve. However, the use of the prosthetic heart valve delivery systems and/or balloon inflation systems described herein may eliminate the need to use a BAV catheter if post dilation is needed, thus saving time, reducing cost, and/or reducing risk to the patient. For example, delivery system 100 (as well as the many variants described herein, including balloon inflation system 300), may be provided with information regarding the ability of the delivery system to be able to expand prosthetic heart valves (e.g., valve 10) to a specified range. For example, this information may be available on tablet 570, which may be supplied as part of the system 100. Thus, if the delivery system 100 is used to deploy prosthetic heart valve 10, and the deployment of the prosthetic heart valve 10 was performed below the upper end of the use range of the delivery system balloon 136, the user may have the ability to add additional volume to the balloon 136 by simply specifying a larger valve size on the computer(s) 560 and/or tablet(s) 570. Once the new parameters are transferred to the balloon inflation system 170 (or balloon inflation system 300, or a similar balloon inflation system), the user may use the balloon actuator 120 (or balloon actuator 120′, 120″, etc.) on the delivery system handle 110 to re-inflate the 136 balloon and expand the prosthetic heart valve 10 further. In some examples, this process may be repeated one or more times to achieve optimal valve performance.

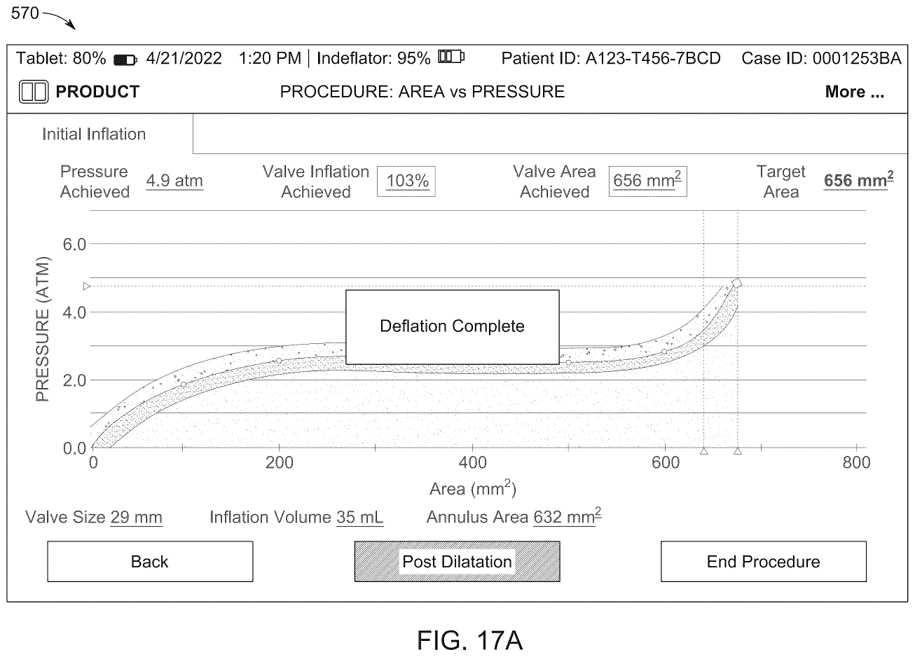

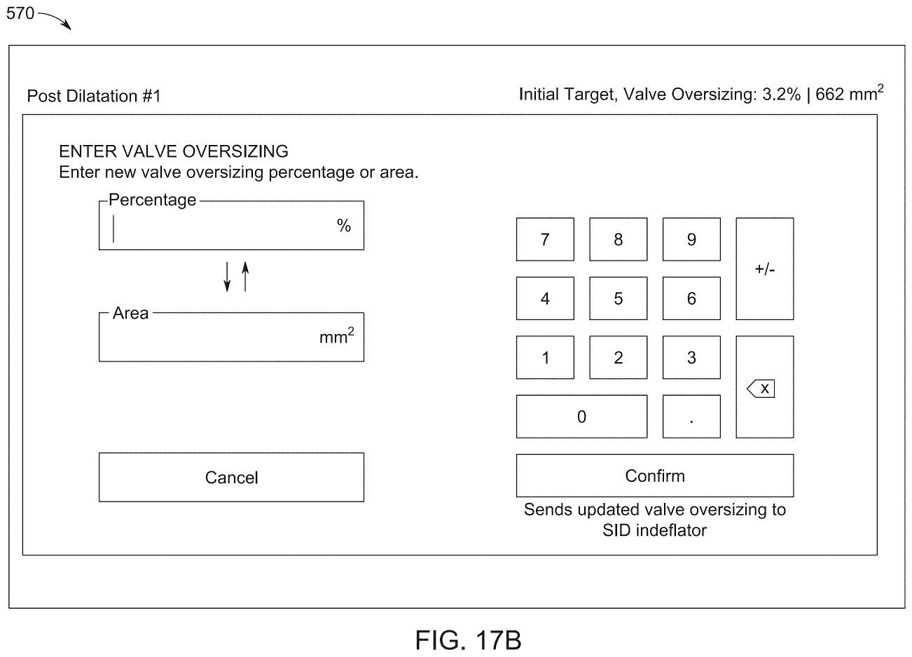

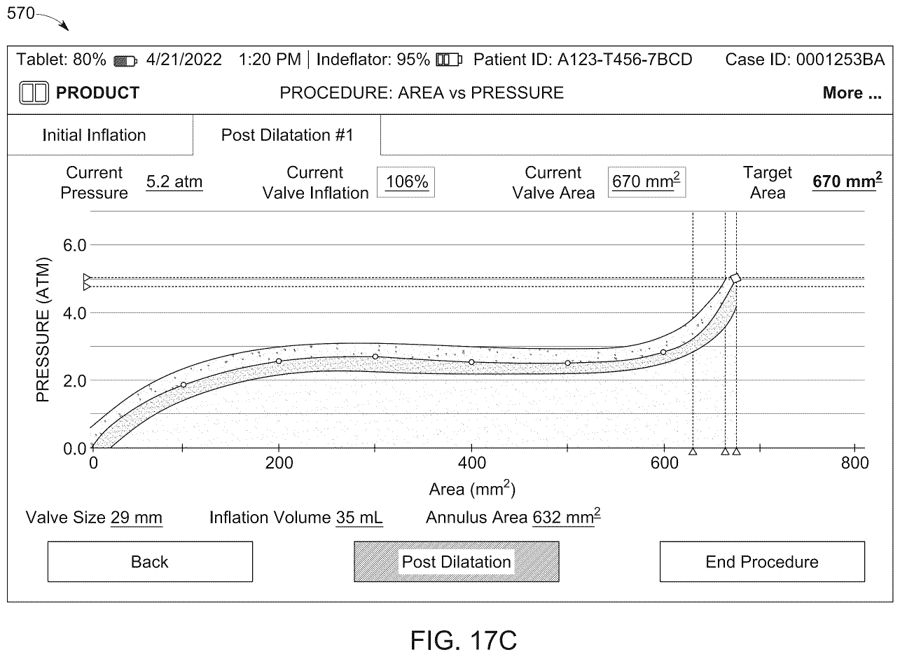

Referring now in addition to FIG. 17A, FIG. 17A shows an exemplary screen that may be displayed, for example on tablet 570, after completion of implanting prosthetic heart valve 10, immediately after the balloon 136 has been deflated. Although the “Deflation Complete” indicator is the primary view in the screen of FIG. 17A, it can also be seen that the valve implantation was performed with 3% oversizing (e.g., 103% valve inflation achieved). In this example, the oversizing correlates to a valve area of 656 mm² being achieved. If, after the balloon 136 has deflated, it is determined that post dilation is desirable, the user has an option to select a post dilation option on the tablet 570 (or alternatively end the procedure if post dilatation is not desired). Selecting the post dilatation option in FIG. 17A may lead the user to the screen displayed in FIG. 17B, in which desired valve oversizing may be entered, either as a percentage oversizing or as an actual area. The valve oversizing percentage or area may be entered on this screen, although it should be within the upper limit of the capabilities of the balloon 136. After entering the valve oversizing values, the user may confirm and then inflate the balloon 136 again, for example by using balloon actuator 120. Because the prosthetic heart valve 10 is not mounted on the balloon 136 during the post dilatation, during the inflation of the balloon 136 in a post dilatation procedure, most or all of the balloon inflation may be performed at a high speed, since the concerns of early balloon inflation are not relevant when the valve is already deployed in the valve annulus. However, in some examples, it may be preferable for at least the end process of the inflation to be relatively slow to provide more data and/or feedback. FIG. 17C shows an example screen displayed on tablet 570 during the actual post dilatation procedure, showing that the 106% valve inflation (compared to the original 103% valve inflation) and an area of 670 mm² (compared to the original 656 mm²) has been achieved. In other words, the screen show in FIG. 17C confirms that the prosthetic heart valve 10 has been further expanded to the desired post dilatation parameters. In some examples, after the balloon 136 deflates, a second (or third, fourth, etc.) post dilatation may be performed if it is determined desirable and the balloon 136 is capable of achieving the desired inflation without risk of bursting or otherwise failing.

The prosthetic heart valve delivery systems (and balloon inflation systems) described herein may not only help with deployment of the prosthetic heart valve 10 and post dilatation procedures, but they may also be leveraged for assisting with system preparation, including de-airing of the system. During use of delivery system 100, the interior fluid line within the balloon catheter and extending into syringe 174 (or syringe 370 or 400) is a closed system. It is generally important that the amount of air within that closed system is minimized. Having air within the system may create problems or potential problems. For example, if the balloon 136 were to burst and air were within the system, air could be released into the blood stream with the potential to cause blockages of blood flow, which could result in a stroke or another medical crisis. Further, air within the system may cause pressure or other readings to be less accurate than if there were no (or a minimum acceptable level of) air within the system. Currently, in order to de-air a balloon catheter prior to use, a manual process is performed in which a vacuum in the balloon catheter is created with a first syringe, and then fluid is pushed into the balloon catheter with a separate syringe, and the cycle is repeated to fill the balloon catheter with fluid and purge air remaining in the balloon catheter. However, in order to confirm that there is no (or a minimal acceptable level of) air in the balloon catheter, a visual check is done which may be a relatively subjective analysis with a potentially high risk of error given the human factor involved. Furthermore, currently, de-airing is typically performed prior to crimping the prosthetic heart valve onto the balloon for delivery. However, prosthetic heart valve delivery system 100 may be provided to the user with prosthetic heart valve 10 pre-crimped over balloon 136, which may make de-airing more difficult. The balloon inflation systems described herein may be used to automate the de-airing process while also creating a more objective, more streamlined, more consistent, and/or more data-based de-airing procedure.

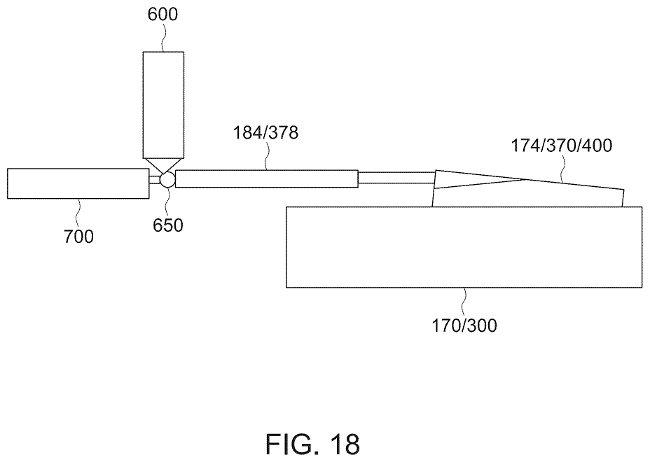

Referring now in addition to FIG. 18, FIG. 18 illustrates a highly schematic example of a setup for a de-airing procedure prior to performing a prosthetic heart valve replacement. In the illustrated example, the user has already received the inflation system 170 (or inflation system 300 or another suitable inflation system), the syringe 174 (or syringe 370 or syringe 400 or another suitable syringe), which may come provided with the fluid line or tubing 184 (or tubing 378 or another suitable tubing) coupled to the handle of a delivery device 700. Delivery device 700 may include, for example, some or all of the components shown in FIG. 4, including a handle 110, introducer 150, catheter 130, and prosthetic heart valve 10, which in some examples may be provided to the user pre-crimped over the balloon 136. In some examples, a rigid plastic tube, such as a loader sheath, may be provided with the delivery device 700. In some example, the loader sheath may already be tightly covering the crimped prosthetic heart valve 10 at the time the system if received by the user, while in other examples, the user may cover the crimped prosthetic heart valve 10 with the loader tube while or prior to beginning the de-airing process.

In some examples, the syringe 174, tubing 184, and delivery device 700 is provided to the user devoid of any liquid within any of the components, for example with the plunger handle 182 of the syringe 174 fully depressed. At the beginning of the de-airing process, if the loader sheath has not already been placed over the prosthetic heart valve 10, the user may cover the prosthetic heart valve 10 with the loader sheath. A three-way stopcock 650 may be provided along the tubing 184 between the syringe 174 and the delivery device 700. The user may place the syringe 174, with the plunger handle 182 fully depressed, into the inflation system 170. The stopcock 650 may be actuated to fluidly disconnect the syringe 174 from the delivery device 700. A reservoir syringe 600, which in some examples may be 60 cc or larger, may be filled with at least 30 ml of contrast mixture (e.g., contrast dye diluted with saline into a 15% contrast mixture) and connected to the stopcock 650. Throughout the de-airing procedure, the reservoir syringe 600 may be held (e.g., manually or via placement on a stand) with the reservoir syringe plunger pointing up (i.e., away from gravity) so that any air within the reservoir syringe 600 will tend to rise to the top of the reservoir syringe 600. At this point, the user may interact with computer 560 and/or tablet 570 (and/or using controls directly on the inflation system(s) 170/300) to actuate the inflation system 170 to withdraw about 30 ml of liquid from the reservoir syringe 600 into (or toward) the syringe 174. During some or all of the de-airing procedure, the syringe 174 may be oriented so that the plunger handle 182 end of the syringe is tilted downward (i.e., in the direction of gravity) relative to the tip of the syringe 174 so that any air within syringe 174 tends to rise toward the tip. At this stage, a mixture of air and fluid is within the syringe 174 and/or the tubing 184. Next, about 10 ml of volume may be purged back from the syringe 174 into the reservoir syringe 600, with any air rising toward the top of reservoir syringe 600. At this point, the user may visually inspect the syringe 174 and the tubing 184 to confirm no air is present. Then, the user may actuate stopcock 650 to fluidly isolate the reservoir syringe 60, and to fluidly connect the syringe 174 with the delivery device 700. It should be understood that stopcock 650 may be a manually actuated stopcock 650, or electronically controlled (e.g., via computer 560 and/or tablet 570 and/or via electronics within the inflation system 170/300) to fluidly isolate and fluidly connect the different portions of the system during the de-airing procedure.

With the syringe 174 fluidly coupled to the delivery device 700, the inflation system 170 may be activated to withdraw about 20 ml of fluid from the delivery device 700, effectively creating a vacuum within the delivery device 700, at which point the stopcock 650 may be actuated to fluidly isolate the delivery device 700 from the syringe 174 and the reservoir syringe 600. The inflation system 170 may then be actuated so that any air that was drawn into the tubing 184 and/or the syringe 174 from the delivery device 700 can be purged into the reservoir syringe 600. This purge may continue until about 20 ml remains in the syringe 174. At this point, the stopcock 650 may be actuated to fluidly isolate the reservoir syringe 600 from the other components. The inflation system 170 may be actuated to push fluid from the syringe 174 into the delivery device 700 to pressurize the balloon 136, for example until the pressure within the balloon 136 is measured at about 3 atm, which may be measured by any of the pressure sensors described above. As the balloon 136 is pressurized, the balloon 136 is prevented from inflating by the overlying loader sheath described above. By pressurizing the balloon 136, any air trapped within the balloon 136 will tend to become compressed, for example with air bubbles reducing in size due to the compression. Then, the inflation system 170 is instructed to withdraw about 20 ml of fluid. As this occurs, any air bubbles within the balloon 136 may more easily leave the balloon 136 and enter the tubing 184 and/or syringe 174 due to the prior compression. After withdrawing the about 20 ml of fluid, the stopcock 650 may be actuated to isolate the delivery device 700 from the other components, and the inflation system 170 may be activated to purge air within the syringe 174 and/or tubing 184 into the reservoir syringe 600. This may continue until about 20 ml of fluid remains in the syringe 174. The stopcock 650 may then be actuated to fluidly isolate the reservoir syringe 600 from the other components, and the inflation system 170 may be activated to push fluid into the balloon 136 until a pressure of about 3 atm is measured, then about 20 ml may be withdrawn back toward the syringe 174, and then fluid may again be pushed into the balloon 136 until a pressure of about 3 atm is measured, at which point the stopcock may be actuated to isolate the delivery device 700.

With the delivery device 700 isolated from the other components, the reservoir syringe 600 may be decoupled from the stopcock 650 and filled with more contrast/saline solution, for example about 40 ml, and then reconnected to the stopcock 650. The inflation system 170 may then be activated to push about 10 ml of fluid from the syringe 174 to the reservoir syringe 600 to purge air within the syringe 174 and/or tubing 184, and then the inflation system 170 may be actuated to withdraw fluid from the reservoir syringe 600 until about 40 ml of solution is within the syringe 174. The user may then actuate the stopcock 650 to fluidly isolate the reservoir syringe 600 from the other components, at which point the inflation system 170 may be instructed to perform a de-airing check. To perform the de-airing check, the inflation system 170 may pull about 2 ml of vacuum. As this occurs, the displacement of the plunger handle 182 of syringe 174 may be compared to the vacuum pressure, and if the relationship between the vacuum pressure and syringe displacement is greater than a pre-defined threshold, the balloon inflation system 170 may notify the user that the de-airing has succeeded. However, if the relationship between vacuum pressure and syringe displacement is below the pre-defined threshold, the balloon inflation system 170 may notify the user that the de-airing has not succeeded, at which point further de-airing may be performed. Once the de-airing is deemed successful, the reservoir syringe 600 may be removed from the stopcock 650 and the delivery device 700 may be ready for use. Additional details regarding the use of the vacuum pressure vs. syringe displacement relationship to detect successful de-airing is described in greater detail in U.S. Patent Application Publication No. 2023/0372097, the disclosure of which is hereby incorporated by reference herein.

It should be understood that, although particular examples of volumes and pressures are described above in connection with the exemplary de-airing procedure, those quantities are merely illustrative and other quantities may be used to achieve the same goal.

Further, it should be understood that various individual features of a prosthetic heart valve delivery system (and/or related methods) are described above. These features and/or methods may be used individually or in combination. For example, any of the above-described features and methods related to (i) inflation system 300 and FIGS. 12A-14, (ii) syringe 400 and FIGS. 15A-15C, (iii) speed inflation profiles and variable-speed inflation and FIGS. 16A-G, (iv) post-dilatation methods and FIGS. 17A-C, and (v) de-airing features and methods and FIG. 18, may each by used individually or in any combination with each other, including all of these features being used as part of a single system and/or method.

Although particular examples of inflation parameters are described above, and although various alternate options are also described, to be clear, it should be understood that the inflation parameters described above represent examples only. In other words, while one example of a three-stage, slow-fast-slow inflation program is described above, this is just one exemplary option. The number of stages of inflation (including one stage or any number of additional stages) may be programmable by a user, and the inflation rates of the one or more steps may be programmable as desired. For example, a three-stage inflation program may include a fast-fast-slow pattern or a medium-fast-slow pattern, or a two-stage inflation program may include a fast-slow pattern or a slow-fast pattern. These additional examples are merely examples, and any other number of stages and inflation speed pattern across the one or more stages may be programmed as desired by the user. Similarly, while the one or more stages of inflation speed may be customized, other parameters of inflation (such as valve oversizing percentage) may be either fixed by the system (e.g. not selectable by the user) or otherwise may be customizable by the user. This also applies to the inflation program, which may be partly or fully customizable, but in other examples may be offered as one or more limited numbers of available fixed programs from which the user may choose. Still further, in some examples above, a pause in inflation is described, for example after an initial slow inflation to allow for a user to analyze the situation before continuing with a fast inflation (including making axial or rotational adjustments after the pause). While one pause (or more pauses) may be programmed into the system between any two stages of inflation, it should be understood that pre-programmed pauses may be partly omitted (e.g. omitted between only some temporally adjacent inflation stages) or entirely omitted, either as part of a fixed pre-programmed inflation pattern or as part of an inflation pattern customized by the user.

As noted above, particularly in connection with FIGS. 17A-C, balloon inflation system 170 or 300 (or variants thereof) may not only be useful for delivery and/or deploying prosthetic heart valve 10 (or variants thereof), but may also be useful in performing post-dilatation after implantation of a prosthetic heart valve (e.g. without needing a separate BAV catheter. In addition to being useful for post-dilatation, the system may similar be useful for pre-dilatation (e.g. balloon valvuloplasty) to prepare the native heart valve (or, in some cases, a previously-implanted prosthetic heart valve) to accept the new prosthetic heart valve. The benefits of using a balloon inflation system 170 or 300 (or variants thereof) for pre-dilatation may be substantially similar to at least some of those described in connection with post-dilatation, including for example the ability to use highly precise inflation while also receiving feedback regarding the inflation.

Although balloon inflation systems 170, 300 are largely described above for use in a prosthetic heart valve implantation procedure, or a related procedure such as balloon valvuloplasty and/or post-dilatation, the balloon inflation systems 170, 300, or at least components and/or technology thereof, may be harnessed for other minimally invasive procedures within the vasculature, including for example percutaneous coronary intervention (“PCI”) such as calcium disruption and/or atherectomy. Both the hardware of the balloon inflation systems 170, 300, as well as the software (including real-time pressure measurements and ability for custom inflation algorithms) may be utilized for more effective calcium disruption and/or atherectomy procedures, examples of which are described in more detail below. It should be understood that, although the calcium disruption described herein is generally described in the context of treating a blood vessel (e.g. a vein, an artery, or any other vessel that carries blood), the calcium disruption may also be performed in substantially the same fashion in other locations, such as within an annulus of a native heart valve, to help disrupt calcium in the native annulus and/or native leaflets associated with the annulus.

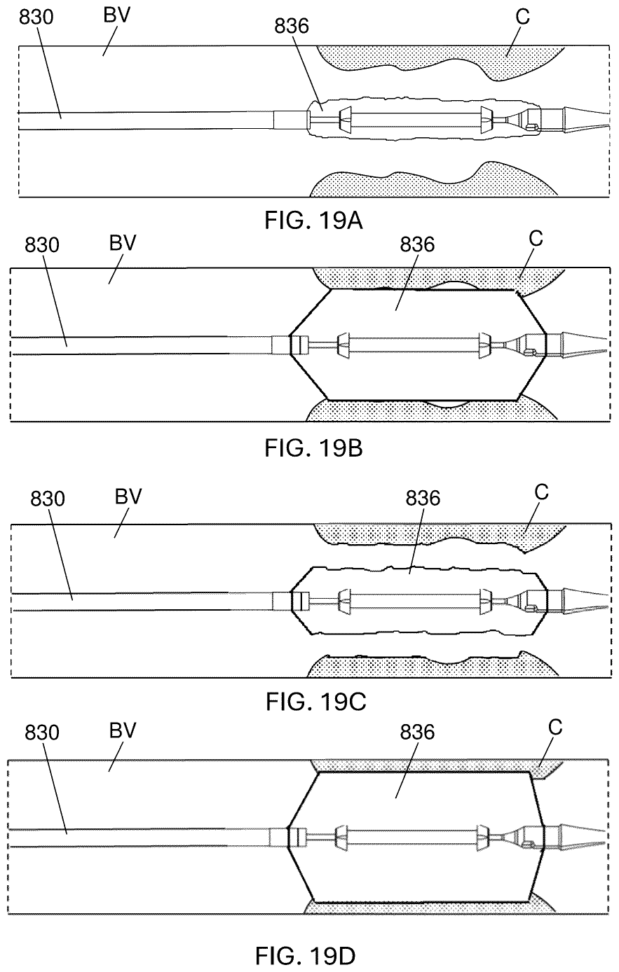

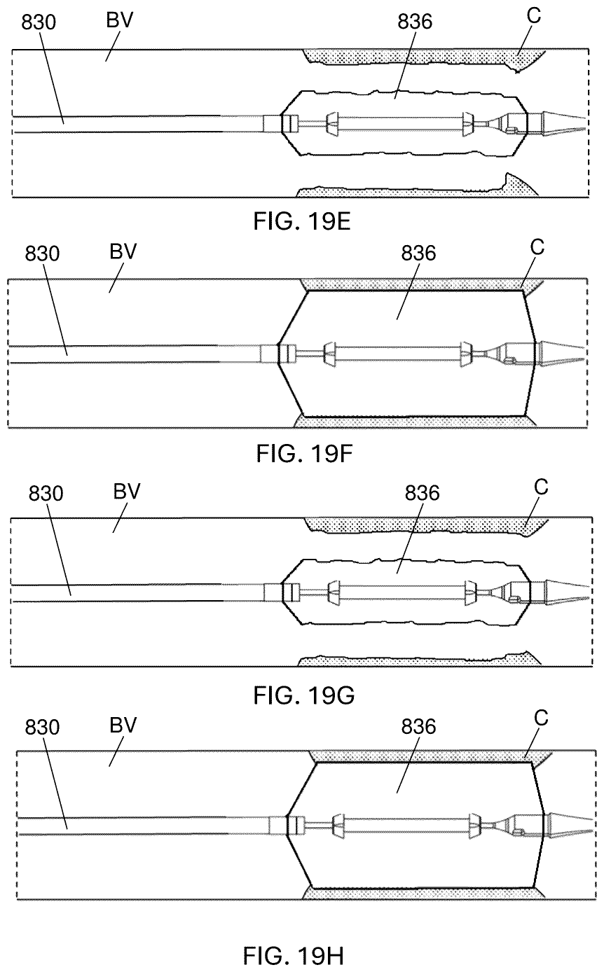

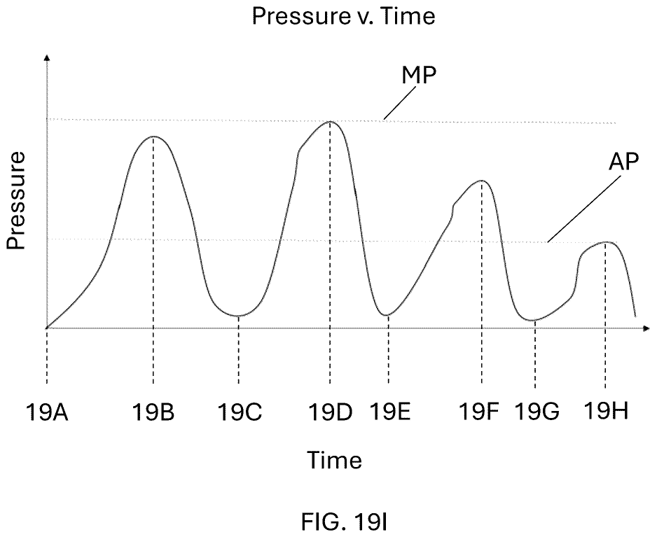

In one example of another use of balloon inflation system 170 or 300 (or variants thereof), an inflatable balloon may be used in a calcium disruption procedure, for example targeting calcium deposits in the coronary arteries and/or peripheral arteries. For example, FIG. 19A illustrates a schematic illustration of a catheter 830 with a balloon 836 mounted thereon (e.g. near a distal end thereof). Catheter 830 and balloon 836 may be identical or otherwise similar to catheter 130 and balloon 136, although in some examples the catheter 830 and balloon 836 may be smaller in size, and may include any other desired features to the particular treatment, including the particular blood vessel BV being targeted. FIG. 19A also schematically illustrates a deposit of calcium C within the blood vessel BV that is the target of the calcium disruption treatment. Although the proximal end of the catheter 830 is not shown in FIG. 19A, it should be understood that the proximal end of the catheter 830 may include a handle, which may be generally similar to handle 110, although certain controls not relevant to the calcium disruption procedure may be omitted. Further, the catheter 830 and/or associated handle may be coupled to a balloon inflation system that is substantially similar or identical to balloon inflation system 170 and/or 300 (with or without additional variations).

FIG. 19A schematically illustrates an early stage in the calcium disruption procedure in which the balloon 836 is in a deflated condition, and the catheter 830 has been intravascularly delivered to the site of the deposit of calcium C in the blood vessel BV. When the balloon 836 has reached the deposit of calcium C, the balloon 836 may be inflated, for example by using a balloon actuator similar or identical to balloon actuator 120 to cause a fluid reservoir or syringe, such as syringe 174, to be activated by the balloon inflation system, such as balloon inflation system 170 or 300, to advance fluid (e.g. saline) into the balloon 836 to inflate the balloon 836. As shown in FIG. 19B, as the balloon 836 inflates, it presses against the deposit of calcium C in an effort to disrupt the calcium C, with the intent to increase the area through which blood can flow.

After the balloon 836 is inflated the first time, as shown in FIG. 19B, the balloon 836 may be maintained in the inflated condition for any desirable amount of time, after which the balloon may be deflated 836, for example by actuating a balloon actuator (e.g. balloon actuator 120) on the handle in an opposite direction to rapidly withdraw fluid from the balloon 836 back toward the fluid reservoir (e.g. syringe 174). This cycle of inflating the balloon 836 and deflating the balloon 836 to press against the deposit of calcium and release the force may be repeated as many times as desired, for example by cycling between manually actuating the balloon actuator in one direction and then the opposite direction in a cyclical fashion. This cycling may be useful to further disrupt the deposit of calcium C which each successive cycle of inflation and deflation until the calcium C has been disrupted to the desired amount and opened the blood vessel BV for blood flow to the desired amount.

Although the description above refers to manually cycling through inflation and deflation of the balloon 836 to disrupt the calcium C, in other examples, the balloon inflation system 170 or 300 (or variants thereof) may be programmed to semi-autonomously or autonomously cycle through any desired custom inflation and deflation algorithm. For example, the balloon size (and/or balloon pressure) to which the balloon 836 is inflated during the inflation phase of the cycle may be targeted based on values input into the balloon inflation system (which targets may be identical for each cycle, or varied among the cycles). Similarly, the speed of the inflation and/or the speed of the deflation may be programmed into the balloon inflation system, and again the inflation speed and/or deflation speed may be the same among the different cycles of inflation/deflation or different among the different cycles of inflation/deflation. The duration with which the balloon 836 is maintained in the inflated state may similarly be programmed into the balloon inflation system, and again the time that the balloon 836 is maintained in the inflated state may be the same among different inflation cycles, or different among different inflation cycles. The duration between the end of one inflation/deflation cycle and the beginning of the next inflation/deflation cycle may similarly be programmed into the balloon inflation system, and again this time may be constant or variable between successive inflation/deflation cycles. The number of total inflation/deflation cycles may also be programmed into the balloon inflation system (but in some examples, the number of inflation cycles may be dependent on data received during the procedure, such as pressures sensed compared to relevant pressure thresholds). Although one or more of these parameters may be set into the balloon inflation system as desired by the user, for breaking up deposits of calcium C, it may be desirable to perform high frequency inflations alternating between low and high pressure to break up the calcium C, simulating a jackhammer type effect. In one example algorithm, at least three parameters may be programmed for a particular treatment procedure, including (i) the number of pulses (e.g. the number of times the balloon is inflated), (ii) the time duration with which the balloon is held in the inflated condition, and (iii) the time duration between successive pulses (e.g. the time in which the balloon is not pressurized between successive pulses). In some examples, the time duration between pulses may be short to create a jackhammer type effect noted above, for example as little as 0.01 seconds or 0.1 seconds, and as large as 1 second, although it should be understood that this is merely one example of possible time duration between successive pulses.

As explained above, a pressure sensor may be used in connection with balloon 836 to determine pressure within the balloon 836 and/or at other locations within the fluid line. For example, a pressure sensor such as pressure sensor 137 described in connection with FIG. 16A may be provided on a shaft interior to the balloon 836, or one or more pressure sensors may alternatively or additional be positioned at other locations within the fluid line, such as within the fluid reservoir (e.g. syringe 174) or in fluid tubing extending from the fluid reservoir.

In some examples, data received from the pressure sensor, which may be substantially real-time data similar to pressure data described elsewhere herein, may be monitored during the cycles of inflation and deflation to determine whether a desired calcium result has been achieved and/or to serve as data that determine if treatment (e.g. pulsing cycles of balloon inflation/deflation) should continue or conclude. Such monitoring could be performed manually, for example by looking at a real-time plot of the pressure waveform on a display, such as computer 560 and/or tablet 570, similar to that shown and described in connection with FIGS. 16A-B, 16D-E, FIG. 17A and/or FIG. 17C. However, in other examples, the pressure data may be monitored autonomously by the balloon inflation system or a computer system operably coupled thereto, with the progress of the treatment being determined, based at least in part, on the autonomously monitored pressure data.

For example, FIGS. 19A-19H schematically show an exemplary succession inflation/deflation cycles, with the balloon being in an initial (deflated) state in FIG. 19A, a first inflation shown in FIG. 19B, a first deflation shown in FIG. 19C, a second inflation shown FIG. 19D, a second deflation shown in FIG. 19E, a third inflation shown in FIG. 19F, a third deflation shown in FIG. 19C, and a final fourth inflation shown in FIG. 19H. In these exemplary figures, the calcium C gets successively disrupted (e.g. compacted and or broken up) with each successive inflation, increasing the available area of flow for blood. It should be understood that the particular number of inflation/deflation cycles shown in FIGS. 19A-19H are merely exemplary, and the progression of the disruption of calcium C is for illustrative purposes only.

FIG. 19I illustrates a graph of pressure versus time, showing an exemplary pressure waveform generated from the cycle of inflation and deflation of FIGS. 19A-19H. For example, the pressure is at or near a minimum while the balloon 836 is deflated and being positioned at the site of the deposit of calcium C before the first inflation, as shown in FIG. 19A. During the first inflation, as shown in FIG. 19B, the balloon 836 contacts the deposit of calcium C, with the measured pressure increasing to a cycle maximum when the balloon 836 is inflated. As the balloon 836 presses against the calcium C, it may compact and/or break up some of the calcium C. Then, the balloon 836 is deflated, as shown in FIG. 19C, with a resulting decrease in measured pressure. During the second cycle, the balloon 836 is inflated again, as shown in FIG. 19D, with a resulting pressure increase being measured as the balloon 836 reaches the inflated state in which it again presses against the deposit of calcium C. Again, the balloon 836 may press against the calcium C and further compact and/or break up the calcium C. In the example of FIG. 19I, the maximum pressure in the second cycle also represents a maximum pressure MP of the entire procedure. The balloon 836 is again deflated, as shown in FIG. 19E, resulting in a pressure decrease as shown in FIG. 19I. During the third cycle of inflation, the balloon 836 is again inflated, as shown in FIG. 19F. Although the balloon 836 may again contact the deposit of calcium C, the calcium C may have ben compacted and/or been broken up enough so that there is less resistance to inflation, resulting in a maximum cycle pressure that is smaller than that in the prior cycle. Then, the balloon 836 may again be deflated, as shown in FIG. 19G, resulting in another pressure drop as shown in FIG. 19I. During the last inflation cycle, the balloon 836 may again be inflated, as shown in FIG. 19H, again resulting in a pressure spike. Once again, although the balloon 836 may again contact the deposit of calcium C, the calcium C may have ben compacted and/or been broken up enough so that there is even less resistance to inflation, resulting in a maximum cycle pressure that is smaller than that in the prior cycle.

Referring still to FIG. 19I, a threshold pressure value, identified on the graph as an acceptable pressure AP. The acceptable pressure AP may represent a threshold pressure value that, once the maximum cycle pressure for an inflation cycle is lower than the threshold pressure value or acceptable pressure AP, it may be determined that a suitable enough of breakup or compaction of the calcium C has been achieved for the procedure. The level of the threshold acceptable pressure AP may be set in any desired fashion. In one particular example, the acceptable pressure AP threshold may be set as a fraction of the maximum pressure MP achieved in earlier cycles. For example, the acceptable pressure AP threshold may be set as about 50%, about 40%, about 30%, about 25%, or any other desired percentage of the maximum pressure MP achieved in earlier cycles. In some examples, the acceptable pressure AP threshold may be set as between about 40% and about 75% of an earlier pressure achieved, which may be the maximum pressure in the initial pulse, or which may be the global maximum pressure in the case that the maximum pressure is achieved in a pulse that is not the initial pulse. In other examples, the acceptable pressure AP threshold may be set as a known value that would be typical of what is seen in a normal or healthy or typical tissue (e.g. blood vessel or heart valve). In other words, in this latter example, if the balloon is inflated and reaches a particular pressure known to correlate to the pressure that would be expected in the case of healthy examples of the particular tissue type, it may be assumed that acceptable calcium dissociation has been achieved. It should be understood that the parameters shown and described in relation to FIG. 19I are merely exemplary, and the waveform may take other shapes. For example, the waveforms may have plateaus as the maximum pressure instead of the sharper peaks shown, and the amount of time between the pulses may be different than shown.

In some examples, the acceptable pressure threshold AP may be set as a fraction of the maximum pressure achieved in the first inflation/deflation cycle, which may be referred to as an initial maximum pressure. In other examples, such as that shown in FIG. 19I, the acceptable pressure threshold AP may be set as a fraction of the global maximum pressure achieved during any inflation/deflation cycle. In some examples, during each inflation cycle, if the maximum pressure achieved is larger than any other maximum pressure achieved in another cycle, the acceptable pressure AP threshold may be updated based on the new global maximum pressure MP achieved during the procedure. In some examples, after multiple procedures have been performed, patient outcomes may be analyzed with respect to the pressure data (which may be stored for later use), for example using machine learning, deep learning, artificial intelligence, etc. to determine what the most effective acceptable pressure threshold AP may be to correlate to the best patient outcomes. It should also be understood that different treatment cycles may be performed on multiple areas of one or more blood vessels BV. For example, after determining that the inflation/deflation cycle of FIGS. 19A-19H has sufficiently treated one deposit of calcium C, the catheter 830 may be advanced or otherwise repositioned to another deposit of calcium C that is to be treated, and the process may be performed again, and this may be repeated as many times as desired. Although examples are described above in which the first maximum pressure of the first pulse is used to determine the acceptable pressure threshold AP, in other examples, the average maximum pressure of the first two pulses, the first three pulses, or any other combination of two or more pulses, may be used in calculating the acceptable pressure threshold AP.

Although different examples are provided above for setting an acceptable pressure threshold AP to determine that the sufficient calcium dissociation or disruption has occurred, still other mechanisms may be used as an indication that sufficient calcium dissociation or disruption occurred. For example, relaxation-type of testing may be performed in which the balloon is inflated to a specific volume, and while the balloon remains at the given inflation volume, the waveform is checked for pressure decrease over time. Calcified anatomies would be expected to have largely consistent radial forces, such that a pressure decrease or decay would not occur (or would not be significant) over time. On the other hand, non-calcified tissue would be expected to stretch at a given pressure. Therefore, decaying pressures would be indicative that the region is more tissue-like (e.g. less calcified). Thus, in some examples, after a pre-selected number of inflation/deflation cycles, the balloon may be inflated with a particular volume of inflation media (or to a particular pressure), with the balloon being maintained in the inflated condition while pressure is monitored. If pressure decay is detected (e.g. a drop in pressure of at least 5%, at least 10%, at least 15% or more), the system may indicate that the tissue has been sufficiently treated and sufficient calcium dissociation or disruption has occurred, which may lead to the user manually terminating the procedure or the system automatically terminating the procedure. On the other hand, if pressure decay is not detected (e.g. a pressure decay of less than 15%, less than 10%, or less than 5%), the system may indicate that treatment should continue, and more inflation/deflation cycles may be performed (manually or automatically), with another pressure decay test being performed after the next inflation/deflection cycle is completed. This process may be continued and/or repeated as desired.

In some examples, a one or more pre-conditioning pulses may be performed in which the balloon is inflated to a low pressure target at the beginning of the treatment to size the vessel being treated (and/or to size the balloon). The goal of the pre-conditioning pulse(s) is to determine the amount of volume of inflation media needed to ensure that the balloon is in contact with the vessel being treated. Then in the subsequent treatment cycles (after the pre-conditioning pulse(s)), a prescribed increase in volume or pressure may be used to dissociate or disrupt the calcium. For example, after the pre-conditioning cycle results in a determination of inflation media needed to contact the vessel, the following treatment cycles may call for increases in inflation media volume by set percentages, such as a 10% increase, a 20% increase, a 30% increase, etc., above the initial inflation media volume determined during the pre-conditioning.

While the example above relates to calcium disruption via rapid inflation/deflation cycling of balloon within the vasculature using a smart balloon inflation system, there are still other applications of the smart balloon inflation system technology described above. Another example is in an atherectomy device.

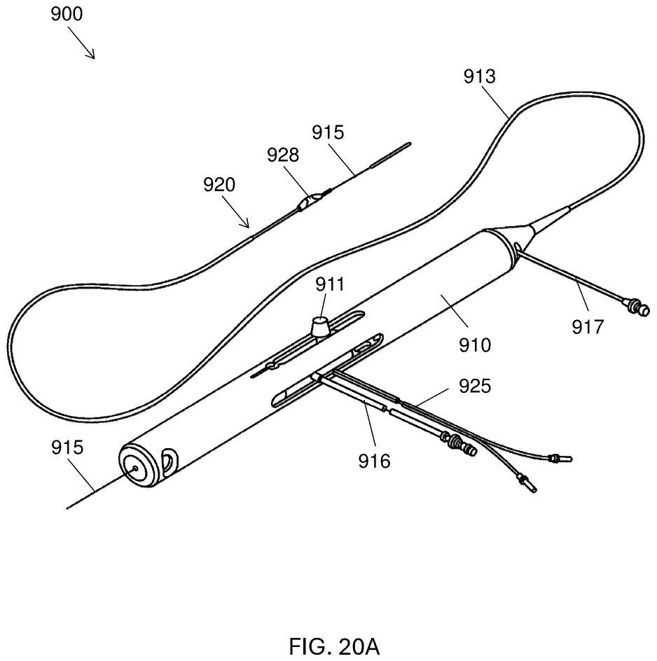

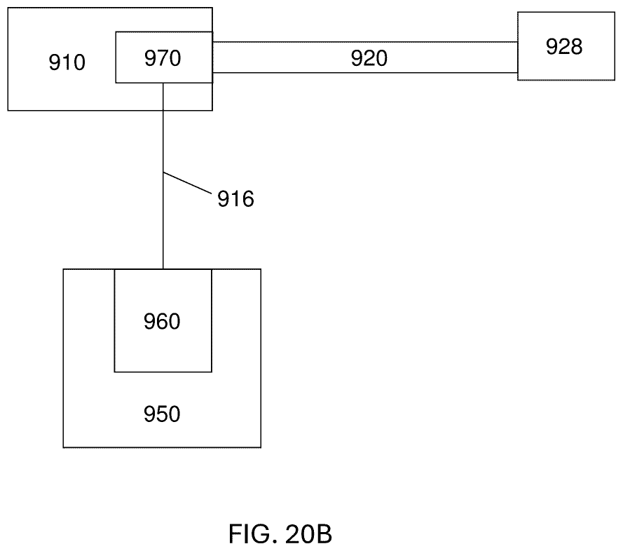

FIG. 20A illustrates one an example of a rotational atherectomy device 900. The device includes a handle portion 910, an elongated, flexible drive shaft 920 having an eccentric enlarged abrading head 928, and an elongated catheter 913 extending distally from the handle portion 910. The drive shaft 920 may be constructed from helically coiled wire and an abrasive element 928 is fixedly attached thereto. Drive shaft 920 may be manufactured from a multi-filar wound coil. The construction of this coil allows for spaces between the filars. These spaces allow fluid, e.g., saline and/or water or other fluid, to pass through to the inner diameter of the drive shaft coil in order to provide cooling and/or lubrication effects to the interface of the drive shaft coil and the guide wire 915.

Continuing with reference to FIG. 20A, the catheter 913 may have a lumen in which most of the length of the drive shaft 920 is disposed, except for the enlarged abrading head 928 and a short section distal to the abrasive element 928. The drive shaft 920 may also contain an inner lumen, permitting the drive shaft 920 to be advanced and rotated over the guide wire 915. A fluid supply line 917 may be provided for introducing a cooling and lubricating solution (typically saline or another biocompatible fluid) into the catheter 913.

The handle 910 may contain a turbine (or similar rotational drive mechanism) for rotating the drive shaft 920 at high speeds. The handle 910 may be connected to a power source, such as fluid (including liquid or compressed air) delivered through a tube 916. A pair of fiber optic cables 925, alternatively a single fiber optic cable may be used, may also be provided for monitoring the speed of rotation of the turbine and drive shaft 920. The handle 910 may include a control knob 911 for advancing and retracting the turbine and drive shaft 920 with respect to the catheter 913 and the body of the handle.

In use, as the rotational drive mechanism is activated, it rotates the drive shaft 920 at high speeds, and thus the abrasive element 928. While the abrasive element 928 is within the vasculature, rotation of the abrasive element 928 (cause by rotation of the drive shaft 920) abrades, sands, or otherwise frictionally interacts with plaque (which may include calcium deposits such as those shown and described in connection with FIGS. 19A-H) to modify (e.g. compress or fracture) the plaque and/or remove the plaque from the wall of the blood vessel in which the abrasive element 928 is rotating. Further details regarding rotational atherectomy devices are described in U.S. Pat. No. 10,413,318, the disclosure of which is hereby incorporated by reference herein.

Although in some examples, the rotational atherectomy device 900 may be powered, at least in part, via compressed air moving through tube 916 to drive a turbine or other rotational drive mechanism to drive the drive shaft 920 and the abrasive element 928, technology of the balloon inflation systems 170 or 300 (or variants thereof) may be incorporated into the rotational atherectomy device 900.

FIG. 20B illustrates a highly schematic illustration of rotational atherectomy device 900, showing a smart inflation system 950, including a fluid reservoir 960, connected to a turbine 970 (or other similar rotational drive mechanism) of handle portion 910 via tube 916. Smart inflation system 950 may be substantially similar or identical to balloon inflation system 170 or 300 (or variants thereof, and including accessory components such as computer 560 and/or table 570), with the main difference being that instead of functioning to pass fluid from a reservoir to an actual balloon for inflation as in balloon inflation systems 170 or 300, smart inflation system 950 functions to pass fluid from reservoir 960 (which may be substantially similar or identical to syringe 174 (or any other syringe described herein or variants thereof) through tube 916 to drive turbine 970 (or a similar rotational drive mechanism) to rotate drive shaft 920 and thus abrasive element 928.

In some examples, the smart inflation system 950, similar to balloon inflation systems 170 or 300 (or variants thereof), is capable of accurately and precisely delivering fluid from the reservoir 960 to the turbine 970 to allow for precise control of the rotation of the drive shaft 920 and abrasive element 928. For example, the power with which the turbine 970 is driven and the frequency of rotation of the turbine 970, drive shaft 920, and/or abrasive element 928 may be manipulated to optimize plaque and/or calcium disruption. In addition to allowing for accurately controlling pressure and/or force outputs from the fluid reservoir 960 via smart inflation system 950, data relating to the pressure and/or force outputs from the smart inflation system 950 may be stored for later analysis. For example, after performing a larger number of atherectomy procedures using smart inflation system 950 to power rotational atherectomy device 900, patient outcomes may be compared to the stored data for each procedure to help relate operating parameters to patient outcomes to help determine which operating parameters result in the best patient outcomes. Such data may be manually analyzed or analyzed using partially or fully automated methods, for example using machine learning, deep learning, artificial intelligence, etc. In some examples, similar to as described in connection with FIG. 19I, the data from the smart inflation system 950 may be monitored during use (e.g. manually, automatically, or semi-autonomously) to determine when an acceptable outcome (e.g. sufficient calcium disruption) has been achieved before completing the procedure (which may include moving the abrasive element 928 to a different position within the blood vessel and repeating the procedure at the new location). Although not separately labeled in FIG. 20B, a pressure sensor similar or identical to the other pressure sensors described herein may be positioned fluidically in contact with the fluid reservoir 960, including for example being positioned within the fluid reservoir 960 or within the tube 916. It should be understood that, although the term smart inflation system is used in connection with component 950, it may also be referred to (as with the other balloon inflation systems described herein) as a smart fluid delivery system.

Some of the techniques described herein, including computer-related and processor-related techniques relating to operation of the inflation system 170 and/or 300 and/or 950, the computer 560, and/or the tablet 570, may be implemented in some examples at least in part by one or more special-purpose computing devices. The disclosure described below may apply to each of the inflation system 170 and/or 300 and/or 950, the computer 560, and/or the tablet 570 as either individual components or components working in unison. The special-purpose computing devices may be hard-wired to perform one or more techniques described herein, including combinations thereof. Alternatively and/or in addition, the one or more special-purpose computing devices may include digital electronic devices such as one or more application-specific integrated circuits (ASICs) or field-programmable gate arrays (FPGAs) that are persistently programmed to perform the techniques. Alternatively and/or in addition, the one or more special-purpose computing devices may include one or more general-purpose hardware processors programmed to perform the techniques described herein pursuant to program instructions in firmware, memory, other storage, or a combination. Such special-purpose computing devices may also combine custom hard-wired logic, ASICs, or FPGAs with custom programming to accomplish the techniques. The special-purpose computing devices may be desktop computer systems, portable computer systems, handheld devices, networking devices, and/or any other device that incorporates hard-wired or program logic to implement the techniques.

FIG. 21 is a block diagram that illustrates a computer system upon which an example may be implemented. The computer system 1000 may include a bus 1002 or other communication mechanism for communicating information, and one or more hardware processors 1004 coupled with bus 1002 for processing information, such as computer instructions and data. The processor/s 1004 may include one or more general-purpose microprocessors, graphical processing units (GPUs), coprocessors, central processing units (CPUs), and/or other hardware processing units.

The computer system 1000 may also include one or more units of main memory 1006 coupled to the bus 1002, such as random-access memory (RAM) or other dynamic storage, for storing information and instructions to be executed by the processor/s 1004. Main memory 1006 may also be used for storing temporary variables or other intermediate information during execution of instructions to be executed by the processor/s 1004. Such instructions, when stored in non-transitory storage media accessible to the processor/s 1004, may turn the computer system 1000 into a special-purpose machine that is customized to perform the operations specified in the instructions. In some embodiments, main memory 1006 may include dynamic random-access memory (DRAM) (including but not limited to double data rate synchronous dynamic random-access memory (DDR SDRAM), thyristor random-access memory (T-RAM), zero-capacitor (Z-RAM™)) and/or non-volatile random-access memory (NVRAM).

The computer system 1000 may further include one or more units of read-only memory (ROM) 1008 or other static storage coupled to the bus 1002 for storing information and instructions for the processor/s 1004 that are either always static or static in normal operation but reprogrammable. For example, the ROM 1008 may store firmware for the computer system 1000. The ROM 1008 may include mask ROM (MROM) or other hard-wired ROM storing purely static information, programmable read-only memory (PROM), erasable programmable read-only memory (EPROM), electrically-erasable programmable read-only memory (EEPROM), another hardware memory chip or cartridge, or any other read-only memory unit.

One or more storage devices 1010, such as a magnetic disk or optical disk, is provided and coupled to the bus 1002 for storing information and/or instructions. The storage device/s 1010 may include non-volatile storage media such as, for example, read-only memory, optical disks (such as but not limited to compact discs (CDs), digital video discs (DVDs), Blu-ray discs (BDs)), magnetic disks, other magnetic media such as floppy disks and magnetic tape, solid-state drives, flash memory, optical disks, one or more forms of non-volatile random-access memory (NVRAM), and/or other non-volatile storage media. The computer system 1000 may be coupled via the bus 1002 to one or more input/output (I/O) devices 1012. For example, the I/O device/s 1012 may include one or more displays for displaying information to a computer user, such as a cathode ray tube (CRT) display, a Liquid Crystal Display (LCD) display, a Light-Emitting Diode (LED) display, a projector, and/or any other type of display.

The I/O device/s 1012 may also include one or more input devices, such as an alphanumeric keyboard and/or any other keypad device. In some examples, the balloon actuators described herein may be an input device. The one or more input devices may also include one or more cursor control devices, such as a mouse, a trackball, a touch input device, or cursor direction keys for communicating direction information and command selections to the processor 1004 and for controlling cursor movement on another I/O device (e.g. a display). A cursor control device typically has degrees of freedom in two or more axes, (e.g. a first axis x, a second axis y, and optionally one or more additional axes z), that allows the device to specify positions in a plane. In some embodiments, the one or more I/O device/s 1012 may include a device with combined I/O functionality, such as a touch-enabled display.

Other I/O device/s 1012 may include a fingerprint reader, a scanner, an infrared (IR) device, an imaging device such as a camera or video recording device, a microphone, a speaker, an ambient light sensor, a pressure sensor, an accelerometer, a gyroscope, a magnetometer, another motion sensor, or any other device that can communicate signals, commands, and/or other information with the processor/s 1004 over the bus 1002.

The computer system 1000 may implement the techniques described herein using customized hard-wired logic, one or more ASICs or FPGAs, firmware, and/or program logic which, in combination with the computer system causes or programs, causes computer system 1000 to be a special-purpose machine. In some examples, the techniques herein are performed by the computer system 1000 in response to the processor/s 1004 executing one or more sequences of one or more instructions contained in main memory 1006. Such instructions may be read into main memory 1006 from another storage medium, such as the one or more storage device/s 1010. Execution of the sequences of instructions contained in main memory 1006 causes the processor/s 1004 to perform the process steps described herein. In alternative embodiments, hard-wired circuitry may be used in place of or in combination with software instructions.

The computer system 1000 may also include one or more communication interfaces 1018 coupled to the bus 1002. The communication interface/s 1018 provide two-way data communication over one or more physical or wireless network links 1020 that are connected to a local network 1022 and/or a wide area network (WAN), such as the Internet. For example, the communication interface/s 1018 may include an integrated services digital network (ISDN) card, cable modem, satellite modem, or a modem to provide a data communication connection to a corresponding type of telephone line. Alternatively and/or in addition, the communication interface/s 1018 may include one or more of: a local area network (LAN) device that provides a data communication connection to a compatible local network 1022; a wireless local area network (WLAN) device that sends and receives wireless signals (such as electrical signals, electromagnetic signals, optical signals or other wireless signals representing various types of information) to a compatible LAN; a wireless wide area network (WWAN) device that sends and receives such signals over a cellular network; and other networking devices that establish a communication channel between the computer system 1000 and one or more LANs 1022 and/or WANs. The network link/s 1020 typically provides data communication through one or more networks to other data devices. For example, the network link/s 1020 may provide a connection through one or more local area networks 1022 (LANs) to one or more host computers 1024 or to data equipment operated by an Internet Service Provider (ISP) 1026. The ISP 1026 provides connectivity to one or more wide area networks 1028, such as the Internet. The LAN/s 1022 and WAN/s 1028 use electrical, electromagnetic, or optical signals that carry digital data streams. The signals through the various networks and the signals on the network link/s 1020 and through the communication interface/s 1018 are example forms of transmission media, or transitory media.

The term “storage media” as used herein refers to any non-transitory media that stores data and/or instructions that cause a machine to operate in a specific fashion. Such storage media may include volatile and/or non-volatile media. Storage media is distinct from but may be used in conjunction with transmission media. Transmission media participates in transferring information between storage media. For example, transmission media includes coaxial cables, copper wire and fiber optics, including traces and/or other physical electrically conductive components that comprise the bus 1002. Transmission media can also take the form of acoustic or light waves, such as those generated during radio-wave and infra-red data communications.

Various forms of media may be involved in carrying one or more sequences of one or more instructions to the processor 1004 for execution. For example, the instructions may initially be carried on a magnetic disk or solid-state drive of a remote computer. The remote computer can load the instructions into its main memory 1006 and send the instructions over a telecommunications line using a modem. A modem local to the computer system 1000 can receive the data on the telephone line and use an infra-red transmitter to convert the data to an infra-red signal. An infra-red detector can receive the data carried in the infra-red signal and appropriate circuitry can place the data on the bus 1002. The bus 1002 carries the data to main memory 1006, from which the processor 1004 retrieves and executes the instructions. The instructions received by main memory 1006 may optionally be stored on the storage device 1010 either before or after execution by the processor 1004.

The computer system 1000 can send messages and receive data, including program code, through the network(s), the network link 1020, and the communication interface/s 1018. In the Internet example, one or more servers 1030 may transmit signals corresponding to data or instructions requested for an application program executed by the computer system 1000 through the Internet 1028, ISP 1026, local network 1022 and a communication interface 1018. The received signals may include instructions and/or information for execution and/or processing by the processor/s 1004. The processor/s 1004 may execute and/or process the instructions and/or information upon receiving the signals by accessing main memory 1006, or at a later time by storing them and then accessing them from the storage device/s 1010.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system for disrupting calcium within a blood vessel, the system comprising:
a handle;
a balloon catheter extending from the handle;
a balloon positioned at a distal end portion of the balloon catheter;
a fluid reservoir in fluid communication with a lumen, the lumen being in fluid communication with an interior volume of the balloon;
a motor operably coupled to the fluid reservoir; and
a pressure sensor fluidically in contact with the fluid reservoir and configured to sense a pressure within the balloon;
wherein the motor is configured to alternately (i) push fluid from the fluid reservoir through the lumen toward the interior volume of the balloon and (ii) withdraw fluid from the interior volume of the balloon toward the fluid reservoir, so as to cycle between balloon inflation and deflation to repeatedly press the balloon against an interior surface of the blood vessel.

2. The system of claim 1, wherein the pressure sensor is positioned interior to the balloon.

3. The system of claim 1, wherein, during operation of the motor to cycle between balloon inflation and deflation, a processor operably coupled to the pressure sensor is configured to monitor pressure data received from the pressure sensor to determine a maximum cycle pressure within the balloon during each successive cycle of balloon inflation and deflation.

4. The system of claim 3, wherein, during operation of the motor to cycle between balloon inflation and deflation, the processor is configured to compare each determined maximum cycle pressure to a threshold pressure value and to indicate that successful calcium disruption has occurred when one of the determined maximum cycle pressures is smaller than the threshold pressure value.

5. The system of claim 4, wherein the threshold pressure value is set to a fraction of a maximum value of all of the determined maximum cycle pressures.

6. The system of claim 4, wherein the threshold pressure value is set to a fraction of the determined maximum cycle pressure within the balloon during a first one of the cycles of balloon inflation and deflation.

7. A method of disrupting calcium within a blood vessel, the method comprising:
inserting a balloon catheter into a patient's vasculature, the balloon catheter being connected to a handle, a balloon being positioned at a distal end portion of the balloon catheter, a fluid reservoir being fluidically coupled to an interior volume of the balloon via a lumen;
advancing the balloon catheter to a treatment site within the blood vessel while the balloon is in a deflated condition;
while the balloon catheter is at the treatment site, actuating a motor of a fluid delivery system to alternately (i) push fluid from the fluid reservoir through the lumen toward the interior volume of the balloon to inflate the balloon into contact with an interior surface of the blood vessel and (ii) withdraw fluid from the interior volume of the balloon toward the fluid reservoir to deflate the balloon, so as to cycle between balloon inflation and deflation to repeatedly press the balloon against the interior surface of the blood vessel to disrupt the calcium at the treatment site.

8. The method of claim 7, wherein a pressure sensor is fluidically in contact with the fluid reservoir, and wherein the method further comprises monitoring pressure within the balloon over time during the cycling between balloon inflation and deflation.

9. The method of claim 8, further comprising determining a maximum cycle pressure within the balloon during each successive cycle of balloon inflation and deflation.

10. The method of claim 9, further comprising comparing each determined maximum cycle pressure within the balloon to a threshold pressure value.

11. The method of claim 10, wherein comparing each determined maximum cycle pressure within the balloon to the threshold pressure value is performed by visually comparing a plot of the monitored pressure within the balloon over time, provided on a display, to the threshold pressure value.

12. The method of claim 10, wherein comparing each determined maximum cycle pressure within the balloon to the threshold pressure value is performed by a processor operably coupled to the fluid delivery system.

13. The method of claim 11, wherein cycling between balloon inflation and deflation is performed at least until the determined maximum cycle pressure within the balloon is lower than the threshold pressure value.

14. The method of claim 10, wherein the threshold pressure value is set to a fraction of a maximum value of all of the determined maximum cycle pressures.

15. The method of claim 10, wherein the threshold pressure value is set to a fraction of the determined maximum cycle pressure within the balloon during a first one of the cycles of balloon inflation and deflation.

16. A system for disrupting calcium within a blood vessel, the system comprising:
a handle;
a catheter extending from the handle;
an abrasive element positioned at a distal end portion of the catheter;
a drive shaft coupled to the abrasive element so that rotation of the drive shaft causes rotation of the abrasive element;
a rotational drive mechanism coupled to the drive shaft so that rotation of the rotational drive mechanism causes rotation of the drive shaft;
a fluid reservoir in fluid communication with a lumen, the lumen being in fluid communication with the rotational drive mechanism so that advancing fluid from the fluid reservoir through the lumen causes rotation of the rotational drive mechanism;
a motor operably coupled to the fluid reservoir; and
a pressure sensor fluidically in contact with the fluid reservoir and configured to sense a pressure within the fluid reservoir;
wherein the motor is configured to push fluid from the fluid reservoir through the lumen to the rotational drive mechanism to rotate the abrasive element.

17. The system of claim 16, wherein the pressure sensor is positioned interior to the fluid reservoir.

18. The system of claim 16, wherein, during operation of the motor to rotate the abrasive element, a processor operably coupled to the pressure sensor is configured to monitor pressure data received from the pressure sensor.
